(19) United States
(12) Patent Application Publication
(10) Pub. No.: US 2026/0311079 A1
(43) Pub. Date: Oct. 8, 2026

(54) AUTOMATED DISAMBIGUATION OF USERS IN A MULTI-TENANT ENVIRONMENT BASED ON SENSOR DATA

(71) Applicant: Panasonic Well LLC, Palo Alto, CA (US)

(72) Inventor: Ramin Firoozye, San Francisco, CA (US)

(21) Appl. No.: 19/276,187

(22) Filed: Jul. 22, 2025

Related U.S. Application Data
(60) Provisional application No. 63/783,809, filed on Apr. 4, 2025.

(51) Int. Cl. G06Q 10/0631; G06F 9/54; G06F 18/2415

(52) U.S. Cl. CPC G06Q 10/06311; G06F 9/542; G06F 18/2415

(57) ABSTRACT

Systems and methods for automatically disambiguating individual users within a multi-tenant environment based on automated evaluation of real-time sensor data obtained within the multi-tenant environment are provided. A user monitoring gateway implemented in a multi-tenant environment processes ambient and direct data from myriad sensors and devices to identify events occurring in the multi-tenant environment. The user monitoring gateway converts this ambient and direct data into an attribute vector having values corresponding to attributes associated with the event and any users detected as being associated with the event. The user monitoring gateway evaluates the attribute vector according to different vectors corresponding to known users to identify the particular users associated with the event.

### CROSS-REFERENCE TO RELATED APPLICATIONS

The present patent application claims the priority benefit of U.S. provisional patent application No. 63/783,809 filed Apr. 4, 2025 and is related to U.S. Patent Applications entitled “SYSTEMS AND METHODS FOR AUTOMATED REVERSE EVENT DETECTION” (Attorney Docket No.: 109304-845022-008200US) filed Jul. 22, 2025, “CONTINUOUS SENSOR MONITORING FOR IDENTIFICATION OF PREDICTIVE ANOMALIES” (Attorney Docket No.: 109304-845021-008300US) filed Jul. 22, 2025 and “DETECTING AND TRANSMITTING EVENTS ASSOCIATED WITH A SUBJECT” (Attorney Docket No.: 109304-845006-008500US) filed Jul. 22, 2025, U.S. Provisional Application 63/783,806 filed Apr. 4, 2025 entitled “SYSTEMS AND METHODS FOR AUTOMATED REVERSE EVENT DETECTION”, U.S. Provisional Application 63/783,800 filed Apr. 4, 2025 entitled “CONTINUOUS SENSOR MONITORING FOR IDENTIFICATION OF PREDICTIVE ANOMALIES” and U.S. Provisional Application 63/783,798 filed Apr. 4, 2025 entitled “DETECTING AND TRANSMITTING EVENTS ASSOCIATED WITH A SUBJECT”, the disclosures of which are incorporated herein by reference in its entirety for all purposes.

### FIELD

The present disclosure relates to systems and methods for automatically disambiguating individual users within a multi-tenant environment based on automated evaluation of real-time sensor data obtained within the multi-tenant environment.

### SUMMARY

Disclosed embodiments provide a framework for automatically identifying and recommending tasks to a member of a task facilitation service in order to reduce the member's cognitive load. According to some embodiments, a computer-implemented method is provided. The computer-implemented method comprises receiving ambient and direct data corresponding to different sensors and devices within a multi-tenant environment. The ambient and direct data is received through a gateway device implemented in the multi-tenant environment. Further, the multi-tenant environment is associated with a set of users. The computer-implemented method further comprises identifying one or more events occurring in the multi-tenant environment. The one or more events are identified based on the ambient and direct data. The computer-implemented method further comprises extracting attribute data from the ambient and direct data. The attribute data corresponds to an individual detected within the multi-tenant environment. Further, the individual is associated with the one or more events. The computer-implemented method further comprises dynamically processing the attribute data through a trained classification algorithm to calculate an attribute vector. The attribute vectors are calculated to disambiguate the set of users according to a set of known attribute vectors associated with the set of users. The computer-implemented method further comprises calculating match probability scores corresponding to the set of users by determining differences between values of the attribute vector and known values corresponding to the set of known attribute vectors. The computer-implemented method further comprises identifying a user from the set of users based on the match probability scores. The match probability score associated with the user satisfies a score threshold value. The computer-implemented method further comprises associating the one or more events with the user. The computer-implemented method further comprises dynamically updating a known attribute vector associated with the user based on the one or more events and feedback corresponding to the one or more events. The updated known attribute vector is used to better associate similar attribute data with the user.

In some embodiments, the computer-implemented method further comprises updating the trained classification algorithm according to the feedback corresponding to the one or more events.

In some embodiments, the attribute data includes physical attribute data, activity data, location data, and timing data corresponding to the one or more events.

In some embodiments, the computer-implemented method further comprises obtaining additional data associated with the user. The additional data is obtained through one or more wearable devices and other personal devices associated with the user. The computer-implemented method further comprises aggregating the additional data and the attribute data.

In some embodiments, the computer-implemented method further comprises dynamically converting new attribute data into a new attribute vector. The new attribute data is extracted from new ambient and direct data obtained within the multi-tenant environment. The computer-implemented method further comprises determining that new match probability scores associated with the new attribute vector fail to satisfy the score threshold value. The computer-implemented method further comprises associating a new event corresponding to the new attribute data with an unknown user.

In some embodiments, the computer-implemented method further comprises generating one or more notifications based on the one or more events and identification of the user. The computer-implemented method further comprises transmitting the one or more notifications according to a set of configurations. The set of configurations is defined based on the identification of the user.

In some embodiments, the different sensors and devices include a set of Internet-of-Things (IoT) devices implemented in the multi-tenant environment. The set of IoT devices is connected to the gateway device through one or more wireless communications channels.

In an embodiment, a system comprises one or more processors and memory including instructions that, as a result of being executed by the one or more processors, cause the system to perform the processes described herein. In another embodiment, a non-transitory computer-readable storage medium stores thereon executable instructions that, as a result of being executed by one or more processors of a computer system, cause the computer system to perform the processes described herein.

Various embodiments of the disclosure are discussed in detail below. While specific implementations are discussed, it should be understood that this is done for illustration purposes only. A person skilled in the relevant art will recognize that other components and configurations can be used without parting from the spirit and scope of the disclosure. Thus, the following description and drawings are illustrative and are not to be construed as limiting. Numerous specific details are described to provide a thorough understanding of the disclosure. However, in certain instances, well-known or conventional details are not described in order to avoid obscuring the description. References to one or an embodiment in the present disclosure can be references to the same embodiment or any embodiment; and, such references mean at least one of the embodiments.

Reference to “one embodiment” or “an embodiment” means that a particular feature, structure, or characteristic described in connection with the embodiment is included in at least one embodiment of the disclosure. The appearances of the phrase “in one embodiment” in various places in the specification are not necessarily all referring to the same embodiment, nor are separate or alternative embodiments mutually exclusive of other embodiments. Moreover, various features are described which can be exhibited by some embodiments and not by others.

The terms used in this specification generally have their ordinary meanings in the art, within the context of the disclosure, and in the specific context where each term is used. Alternative language and synonyms can be used for any one or more of the terms discussed herein, and no special significance should be placed upon whether or not a term is elaborated or discussed herein. In some cases, synonyms for certain terms are provided. A recital of one or more synonyms does not exclude the use of other synonyms. The use of examples anywhere in this specification including examples of any terms discussed herein is illustrative only, and is not intended to further limit the scope and meaning of the disclosure or of any example term. Likewise, the disclosure is not limited to various embodiments given in this specification.

Without intent to limit the scope of the disclosure, examples of instruments, apparatus, methods and their related results according to the embodiments of the present disclosure are given below. Note that titles or subtitles can be used in the examples for convenience of a reader, which in no way should limit the scope of the disclosure. Unless otherwise defined, technical and scientific terms used herein have the meaning as commonly understood by one of ordinary skill in the art to which this disclosure pertains. In the case of conflict, the present document, including definitions will control.

Additional features and advantages of the disclosure will be set forth in the description which follows, and in part will be obvious from the description, or can be learned by practice of the herein disclosed principles. The features and advantages of the disclosure can be realized and obtained by means of the instruments and combinations particularly pointed out in the appended claims. These and other features of the disclosure will become more fully apparent from the following description and appended claims, or can be learned by the practice of the principles set forth herein.

### BRIEF DESCRIPTION OF THE DRAWINGS

FIG. 1 shows an illustrative example of an environment in which a user monitoring gateway implemented in a multi-tenant environment dynamically processes ambient and direct data associated with different sensors and devices to disambiguate users within the multi-tenant environment in accordance with at least one embodiment;

FIG. 2 shows an illustrative example of an environment in which a user monitoring gateway aggregates attribute data for generation of one or more attribute vectors for identification of different users within a multi-tenant environment in accordance with at least one embodiment;

FIG. 3 shows an illustrative example of an environment in which a data collection module implemented by a user monitoring gateway aggregates attribute data according to different attribute categories for calculation of attribute vectors and identification of users within a multi-tenant environment in accordance with at least one embodiment;

FIG. 4 shows an illustrative example of an environment in which a user identification engine implemented by a user monitoring gateway dynamically processes attribute vectors corresponding to received ambient and direct data within a multi-tenant environment to identify a possible user associated with a detected event in accordance with at least one embodiment;

FIG. 5 shows an illustrative example of a process for aggregating attribute data corresponding to one or more possible users within a multi-tenant environment in response to detecting an event within the multi-tenant environment in accordance with at least one embodiment;

FIG. 6 shows an illustrative example of a process for generating attribute vectors based on received attribute data corresponding to a detected event and for identification of a user associated with the detected event in accordance with at least one embodiment;

FIG. 7 shows an illustrative example of a process for evaluating an attribute vector corresponding to a detected event against a set of known attribute vectors corresponding to known users within a multi-tenant environment to associate the detected event with a particular user in accordance with at least one embodiment;

FIG. 8 shows an illustrative example of an environment in which communications with members are processed in accordance with at least one embodiment; and

FIG. 9 shows a computing system architecture including various components in electrical communication with each other using a connection in accordance with various embodiments.

In the appended figures, similar components and/or features can have the same reference label. Further, various components of the same type can be distinguished by following the reference label by a dash and a second label that distinguishes among the similar components. If only the first reference label is used in the specification, the description is applicable to any one of the similar components having the same first reference label irrespective of the second reference label.

### DETAILED DESCRIPTION

In the following description, for the purposes of explanation, specific details are set forth in order to provide a thorough understanding of certain inventive embodiments. However, it will be apparent that various embodiments may be practiced without these specific details. The figures and description are not intended to be restrictive. The word “exemplary” is used herein to mean “serving as an example, instance, or illustration.” Any embodiment or design described herein as “exemplary” is not necessarily to be construed as preferred or advantageous over other embodiments or designs.

Disclosed embodiments may provide a framework to automatically identify and recommend tasks to a member of a task facilitation service in order to reduce the member's cognitive load. Through this framework, the task facilitation service can monitor communications between a member and an assigned representative to automatically identify possible tasks that can be performed for the benefit of the member. Further, the task facilitation service can automatically identify any additional information that may be required for the creation of these tasks. Once these tasks have been created, the task facilitation service can coordinate with the representative and/or third-party services to perform these tasks for the benefit of the member.

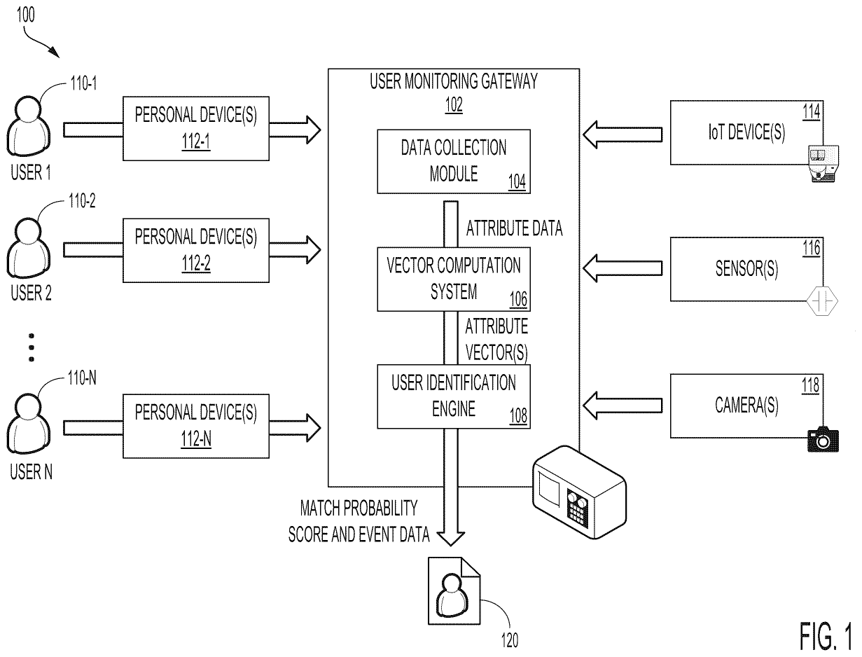

FIG. 1 shows an illustrative example of an environment 100 in which a user monitoring gateway 102 implemented in a multi-tenant environment dynamically processes ambient and direct data associated with different sensors and devices to disambiguate users 110-1-110-N within the multi-tenant environment in accordance with at least one embodiment. In the environment 100, a user monitoring gateway 102 may be implemented to dynamically, and in real-time or near real-time, monitor ambient and direct data from different devices and sensors implemented within a multi-tenant environment to detect events associated with different users 110-1-110-N within the multi-tenant environment. For instance, in an embodiment, the user monitoring gateway 102 is configured and implemented to automatically process ambient and direct data from myriad devices and sensors within a multi-tenant environment to track the health and wellbeing of different users 110-1-110-N within the multi-tenant environment. As an illustrative example, the user monitoring gateway 102 may continuously process ambient and direct data from the myriad devices and sensors within the multi-tenant environment to detect any medical emergencies and accordingly transmit notifications to entities (e.g., caregivers, emergency responders, medical professionals, etc.) that may be able to address these medical emergencies. Further, the user monitoring gateway 102 may continuously process the ambient and direct data from the myriad devices and sensors to monitor user activities within the multi-tenant environment. For instance, the user monitoring gateway 102 may process this ambient and direct data to dynamically track user movements and location within the multi-tenant environment, track user activities within the multi-tenant environment (e.g., eating, exercising, performing hygienic tasks, sleeping, etc.), and the like.

Ambient data, in an embodiment, includes data collected from the myriad devices and sensors within the multi-tenant environment and that is not specific to any given user within the multi-tenant environment. For example, ambient data may include temperature and humidity readings obtained from different IoT devices 114 and/or sensors 116 implemented within the multi-tenant environment and corresponding to the temperature and humidity within different areas within the multi-tenant environment (e.g., different rooms, etc.). As another illustrative example, ambient data may include images or other audiovisual media collected through one or more cameras 118 implemented within the multi-tenant environment. These images or other audiovisual media may not be specific to any user within the multi-tenant environment and may denote a current representative state of the multi-tenant environment.

Direct data, in an embodiment, includes data that is specifically associated with a user within the multi-tenant environment. For example, for a given user, direct data may include biometric readings obtained through personal devices (e.g., glucose monitoring devices, blood pressure reading devices, electrocardiogram monitoring devices, personal fitness monitoring devices, embedded devices, etc.) associated with the user. As another illustrative example, direct data may include data collected from the myriad devices and sensors within the multi-tenant environment that is specific to a particular user. For example, if a camera implemented within the multi-tenant environment captures images and/or other audiovisual media corresponding specifically to the user (e.g., the camera detects the user entering the multi-tenant environment and begins capturing images and/or audiovisual media associated with the user), these images and/or other audiovisual media may constitute direct data associated with the user.

In an embodiment, the user monitoring gateway 102 transmits event data corresponding to a particular event detected within the multi-tenant environment and associated with a particular user to different entities based on the type or category of the particular event. The event data may be transmitted to these different entities based on a set of preferences corresponding to the particular user associated with the event. Returning to an illustrative example described above, if the particular event corresponds to a medical emergency associated with a user, the user monitoring gateway 102 may evaluate the user preferences associated with the user and determine that the data associated with the particular event (e.g., medical emergency) is to be transmitted to emergency responders which may be deployed to address the medical emergency. As another illustrative example, if the particular event corresponds to a user's lack of appetite (e.g., the user's eating habits have changed over a period of time, as detected by the user monitoring gateway 102), the user monitoring gateway 102 may evaluate the user preferences associated with the user and determine that the data associated with the particular event is to be transmitted to a caregiver associated with the user. The caregiver may use this data to further evaluate the user's eating habits and attend to the user to address the user's lack of appetite.

In some instances, the user monitoring gateway 102 may transmit event data corresponding to any detected events within the multi-tenant environment to a remote user monitoring system (not shown) that may broadcast the event data to different instances of a user health monitoring application implemented on different computing devices. The remote user monitoring system may maintain different user records corresponding to different users associated with the remote user monitoring system (e.g., users 110-1-110-N, other users in other multi-tenant environments, etc.) through which event data associated with these different users may be tracked and stored. The remote user monitoring system, in some instances, may provide user monitoring services for various users within different environments. For example, the remote user monitoring system may automatically coordinate with different user monitoring gateways (such as user monitoring gateway 102) to detect any user anomalies that may be indicative of different issues related to a user (e.g., a medical emergency, a new medical condition, a need for assistance, etc.). Further, the remote user monitoring system may automatically coordinate with different user monitoring gateways to dynamically track user activities within these different environments to generate user baselines that may be used to detect, in real-time or near real-time, the aforementioned user anomalies. In some instances, the remote user monitoring system may further coordinate communication with identified entities according to user preferences as defined during an onboarding process and/or through interaction with the user health monitoring application or with a web portal implemented by the remote user monitoring system.

In a multi-tenant environment, where various users may be dynamically monitored in real-time or near real-time through different devices and sensors within the multi-tenant environment, ambient and direct data collected within the multi-tenant environment needs to be disambiguated to associate portions of the ambient and direct data with the appropriate user 110-1-110-N within the multi-tenant environment. Thus, if the user monitoring gateway 102 is implemented in a multi-tenant environment (such as the environment 100), the user monitoring gateway 102 is configured with various components, as described herein, for disambiguating any received ambient and direct data to associate corresponding events with the appropriate users 110-1-110-N within the multi-tenant environment. Further, once data associated with a particular event has been associated with a particular user from the users 110-1-110-N, the user monitoring gateway 102 may transmit the event data to the remote user monitoring system described above and/or to particular entities as designated by the particular user. As described in greater detail herein, the event data generated and transmitted by the user monitoring gateway 102 may be encrypted or otherwise obfuscated to ensure user privacy, whereby only authorized entities provided with corresponding credentials (e.g., cryptographic keys, cryptographic tokens, etc.) may use these credentials to decrypt or otherwise access the event data associated with a particular user. This encryption or obfuscation of event data may be performed subject to any applicable policies or regulations (e.g., Health Insurance Portability and Accountability Act (HIPAA), local privacy regulations, etc.).

The user monitoring gateway 102, in an embodiment, is a standalone computing device that is configured to interact with different devices and sensors through corresponding communications channels to obtain and process ambient and direct data within a multi-tenant environment to detect and categorize different events occurring within the multi-tenant environment. For example, as illustrated in FIG. 1, the user monitoring gateway 102 may be connected to one or more Internet-of-Things (IoT) devices 114 within the multi-tenant environment. These connections (i.e., communications channels) may be established according to one or more IoT protocols. Example IoT protocols that may be implemented to facilitate the connections between the myriad IoT devices 114 within the multi-tenant environment and the user monitoring gateway 102 may include, but are not limited to, the Advanced Message Queuing Protocol (AMQP), Bluetooth®, Bluetooth® Low Energy (BLE), Adaptive Network Topology (ANT), ANT+, Zigbee, Z-Wave, Narrowband Internet of Things (NB-IoT), Matter, the Constrained Application Protocol (CoAP), the Data Distribution Service (DDS) protocol, LoRaWAN, Lightweight Machine-to-Machine (LWM2M) protocol, Message Queuing Telemetry Transport (MQTT), Wi-Fi, Near-Field Communication (NFC), and the like. IoT devices 114 that may be implemented in a multi-tenant environment may include, but are not limited to, home security devices (e.g., doorbell cameras, smart locks, alarm systems, etc.), home appliances (e.g., smart refrigerators, smart lights, smart televisions, smart speakers, smart washers and dryers, etc.), and the like.

In some instances, one or more IoT devices 114 may be physically connected to the user monitoring gateway 102, such as through wired connections through which wired communications may be transmitted between these one or more IoT devices 114 and the user monitoring gateway 102. If the user monitoring gateway 102 is physically connected to one or more IoT devices 114 within the multi-tenant environment, location information obtained from these one or more IoT devices 114 may be used to help disambiguate different users 110-1-110-N within the multi-tenant environment. For example, if the user monitoring gateway 102 is physically connected to a wired motion detection device within a user's room, the user monitoring gateway 102 may automatically associate data from the wired motion detection device with the user if the wired motion detection device is triggered at times during which the user is known to be present within the room. In some instances, the user monitoring gateway 102 may automatically apply one or more data filters to data collected from a wired IoT device, which may prevent associating data that is irrelevant to a user with that user. Returning to the earlier illustrative example of a wired motion detection device physically connected to the user monitoring gateway 102, the user monitoring gateway 102 may apply a data filter that may prevent association of motion detection data collected at certain times that coincide with other users entering or performing various functions within the room (e.g., a caregiver performing their regular visit to the room, a registered guest visiting the room, a roommate being present within the room, etc.). The application of such a data filter may reduce the likelihood of data collected from the wired IoT device being erroneously associated with different users during periods of increased traffic within the room.

As illustrated in FIG. 1, the user monitoring gateway 102 may additionally be connected to one or more sensors 116 within the multi-tenant environment and through which the user monitoring gateway 102 may dynamically obtain ambient and direct data. These one or more sensors 116 may include, but are not limited to, home temperature sensors, air quality sensors, water monitoring sensors (e.g., leak detectors, water quality sensors, etc.), motion sensors, light sensors, door and window sensors, and the like. The one or more sensors 116 may be connected to the user monitoring gateway 102 through one or more communications protocols, including any of the IoT protocols described above or other available communications protocols. For instance, the one or more sensors 116 may be implemented on one or more of the IoT devices 114 described above (e.g., a light sensor implemented on a smart television to determine a desired television brightness, a door sensor implemented on a smart refrigerator for detecting when the smart refrigerator is accessed, an air quality sensor implemented on a smart air conditioning/heating system, etc.). Thus, in some instances, the ambient and direct data obtained from one or more sensors 116 may be included within the ambient and direct data obtained from one or more IoT devices 114. In some instances, the ambient and direct data from one or more sensors 116 may be provided through standalone communications channels established between the user monitoring gateway 102 and these one or more sensors 116.

The user monitoring gateway 102 may further obtain ambient and direct data through one or more cameras 118 implemented within the multi-tenant environment. These cameras 118 may continuously, or in response to a triggering event, collect audiovisual data (e.g., audial and voice data, images, video captures, etc.) within and without the multi-tenant environment. For example, the multi-tenant environment may include a doorbell camera that may automatically record audiovisual data when a user is detected within a pre-defined range of the doorbell camera and/or when a user interacts with the doorbell camera. As another illustrative example, the multi-tenant environment may include one or more cameras within different rooms that may automatically record audiovisual data when a user within the multi-tenant environment enters any of these different rooms. In some instances, a camera within the multi-tenant environment may be configured to automatically record any audiovisual data in the event of any sudden change in movement of a detected user within range of the camera. For example, if a camera detects a user within a particular room and the user suddenly falls to the ground (e.g., slips, faints, etc.), the camera may automatically detect this sudden fall and record the user. In some instances, the one or more cameras 118 within the multi-tenant environment may transmit any recorded audiovisual data to the user monitoring gateway 102 continuously or in response to a triggering event (such as the triggering events described above). The one or more cameras 118 may be connected to the user monitoring gateway 102 through one or more communications protocols, including but not limited to HyperText Transfer Protocol (HTTP) Live Streaming (HLS), Real-Time Streaming Protocol (RTSP), Real-Time Messaging Protocol (RTMP), Dynamic Adaptive Streaming over HTTP (MPEG-DASH), Web Real-Time Communication (WebRTC), Secure Reliable Transport (SRT), Session Initiation Protocol (SIP), and the like. In some instances, similar to the one or more sensors 116, the one or more cameras 118 may be implemented on one or more of the IoT devices 114 described above. Alternatively, one or more cameras 118 may be standalone devices through which standalone communications channels may be established with the user monitoring gateway 102.

It should be noted that the example devices and sensors 114-118 are provided for illustrative purposes and the user monitoring gateway 102 may obtain ambient and direct data from additional and/or alternative devices and sensors implemented within the multi-tenant environment. For example, the user monitoring gateway 102 may be connected to one or more ambient microphones or audial sensors that may continuously detect and transmit audial recordings corresponding to different audial signals detected within the multi-tenant environment. As another illustrative example, the user monitoring gateway 102 may be connected to one or more gaming devices that may dynamically monitor user inputs to one or more gaming control devices to determine what actions may be performed within a gaming environment. These user inputs may be used by the user monitoring gateway 102 to determine a user's hand-eye coordination (or lack thereof) and identify any events should the user's hand-eye coordination become impaired (as determined through evaluation of the user inputs).

In an embodiment, the user monitoring gateway 102 further obtains, in real-time or near real-time, ambient and direct data from personal devices 112-1-112-N associated with users 110-1-110-N, respectively. The personal devices 112-1-112-N may include, but are not limited to, personal fitness tracking devices, smartphones, smart watches, tablet computing devices, smart eyewear, augmented reality devices, wearable medical devices (e.g., glucose level monitors, blood pressure monitors, personal thermometers, electrocardiogram monitors, pacemakers, etc.), biosensors, and the like. In some instances, the personal devices 112-1-112-N may be implemented within the multi-tenant environment but are otherwise configured to generate direct data corresponding to the users 110-1-110-N within the multi-tenant environment according to known user characteristics (e.g., biometric information, known movement routines, etc.). The personal devices 112-1-112-N, in some example, may be on the corresponding users 110-1-110-N, respectively (e.g., wearable devices, embedded devices, etc.). The personal devices 112-1-112-N may further include different location-based devices that may be implemented in user-specific locations, such as in a user's furniture (bed, chair, etc.) or immediate environment (e.g., room, etc.). These location-based devices may dynamically collect ambient and direct data that may be associated with a given user. These personal devices 112-1-112-N may be connected to the user monitoring gateway 102 through any of the communications protocols (wired and/or wireless) described herein. In an embodiment, when a user establishes a communications channel between a user's personal device and the user monitoring gateway 102, the user monitoring gateway 102 automatically associates the user's personal device with the particular user to allow for enhanced disambiguation of ambient and direct data from the user's personal device, as it may be more likely that any ambient and direct data obtained from the user's personal device may be associated with the user. However, this does not prevent the user monitoring gateway 102 from evaluating the ambient and direct data to determine whether the ambient and direct data is associated with a different user within the multi-tenant environment (e.g., another user authorized to use the personal device, another user interacting with the owner of the personal device and detected by the personal device, etc.).

In an embodiment, the user monitoring gateway 102 implements a data collection module 104 that continuously, and in real-time or near real-time, obtains ambient and direct data associated with the multi-tenant environment from the various personal devices 112-1-112-N associated with the different users 110-1-110-N and from any IoT devices 114, sensors 116, and cameras 118 implemented in the multi-tenant environment. The data collection module 104 may comprise one or more computer systems of the user monitoring gateway 102 or may be implemented as an application or process executing on the user monitoring gateway 102. In some instances, the data collection module 104 may be implemented through various special-purpose components of the user monitoring gateway 102 that can facilitate real-time or near real-time collection and processing of different ambient and direct data from any number of different sensors and devices within the multi-tenant environment and as the ambient and direct data from these different sensors and devices is received.

In an embodiment, the data collection module 104 implements a set of libraries or other resources corresponding to different data formats utilized by different sensors and devices compatible with the user monitoring gateway 102. For instance, the ambient and direct data generated and transmitted by the myriad sensors and devices within a multi-tenant environment and connected to the user monitoring gateway 102 through different communications channels may be constructed using different data structures. The data collection module 104 may maintain a set of libraries or other resources that indicate these different data structures according to the types of devices and sensors from which the user monitoring gateway 102 may be connected to. For instance, the data collection module 104 may include one or more JavaScript Object Notation (JSON) libraries, Protocol Buffers (Protobuf), MQTT, Matter, BLE, Zigbee, ZWave, Controller Area Network (CAN) bus, Concise Binary Object Representation (CBOR) libraries, Binary JSON (BSON) libraries, Extensible Markup Language (XML) libraries, Comma-Separated Values (CSV) libraries, and the like. Additionally, the data collection module 104 may implement a set of libraries or other resources for processing semi-structured and unstructured data from different sensors and devices within the multi-tenant environment. For instance, the data collection module 104 may implement computer vision, computer audition, or other machine learning algorithm that is dynamically trained to process audiovisual data from different sensors and devices within the multi-tenant environment to automatically identify various characteristics associated with the audiovisual data (e.g., user(s) within a room, voice signatures, etc.). As another illustrative example, the data collection module 104 may receive telemetry data or other IoT output from the one or more IoT devices 114 that is unstructured and may necessitate format-specific algorithms or other processes for evaluating the telemetry data or other IoT output.

In an embodiment, the data collection module 104 is configured to dynamically process the ambient and direct data from the myriad sensors and devices within the multi-tenant environment according to a set of different attribute data categories. For example, the data collection module 104 may dynamically process the collected ambient and direct data to detect any physical attributes associated with any user detected within the multi-tenant environment. These physical attributes may include, for instance, the physical appearance of a detected user such as the detected user's facial attributes (e.g., facial structure and shape, dental structure, eye color, hair color, facial hair, etc.) and bodily attributes (e.g., body structure and type, skin color, hair color, etc.). The physical attributes associated with the user and detected by the data collection module 104 may further include the user's weight and height, which may be determined through sensor data (e.g., measurements from a smart scale, etc.) or through other approximation (e.g., visual, audial, infrared, mmWave, LIDAR, etc.) by processing received audiovisual data using computer vision or other machine learning algorithm trained to process visual images or other data to identify and approximate height and weight data associated with any detected user. The data collection module 104 may additionally identify, from the ambient and direct data, a detected user's movement or gait within the multi-tenant environment. For instance, by processing the received audiovisual data using computer vision or other machine learning algorithm, the data collection module 104 may collect various data points corresponding to a detected user's position within a room that may be aggregated to define the detected user's movement and gait within the room.

In some instances, the data collection module 104 may further process any obtained audial data using Natural Language Processing (NLP) or other voice-recognition machine learning algorithm to generate a voice signature of any detected user within the multi-tenant environment. The voice signature may correspond to different characteristics associated with the user's voice that may be used to uniquely identify the user based on their audial communications. As each user may have a unique voice signature, detection of a voice signature corresponding to a particular user may increase the likelihood that a detected event is associated with the particular user.

From the ambient and direct data, the data collection module 104 may further detect any location attributes corresponding to any user detected within the multi-tenant environment. These location attributes may include, for instance, the absolute location of a detected user within the multi-tenant environment at a given time. An absolute location may, for instance, correspond to a particular room or other portion of the multi-tenant environment. In response to receiving ambient and direct data from the different sensors and devices within the multi-tenant environment, the data collection module 104 may process the ambient and direct data to detect which room or other portion of the multi-tenant environment the ambient and direct data originated from. In some instances, the ambient and direct data may correspond to a sequence of events that may denote user movement within the multi-tenant environment. For instance, ambient and direct data collected from different devices and sensors within different locations of the multi-tenant environment and obtained over a short period of time may denote movement of a particular user through these different locations. This detected movement may be commonly associated with a particular user and, thus, may be used by the data collection module 104 to identify the user associated with the movement.

If, through processing of the ambient and direct data, the data collection module 104 detects that a user is associated with the ambient and direct data, the data collection module 104 may associate the detected user with the location from which the corresponding ambient and direct data was obtained (e.g., the absolute location). In addition to determining the absolute location of a detected user, the data collection module 104 may process the received ambient and direct data to detect the relative location of the detected user over a period of time. For instance, if a detected user is moving between different rooms within the multi-tenant environment and two distinct sensors and/or devices detect the user within these different rooms over a short period of time, the data collection module 104 may process the ambient and direct data corresponding to these two distinct sensors and/or devices to detect the user's transition from one room to another and, thus, determine the user's relative location within the multi-tenant environment.

The location attributes may further include any geolocation data corresponding to a detected user. This geolocation data may include, for instance, Global Positioning System (GPS) coordinates obtained from sensors and/or devices that implement a GPS receiver or antenna that allows for continual communication with a GPS constellation to obtain geolocation data. The geolocation data may be obtained from one or more user's personal devices and/or from sensors/devices implemented within the multi-tenant environment. This geolocation data may provide a more granular location of a detected user within the multi-tenant environment. While GPS is used extensively throughout the present disclosure for the purpose of illustration, geolocation data may be obtained through other techniques. For instance, geolocation data may be obtained through, but not limited to, Globalnaya Navigatsionnaya Sputnikovaya Sistema (GLONASS), the BeiDou Navigation Satellite System (BDS), the Galileo navigation system, and the like.

In some instances, the location attributes may further include location proximity data that represents a detected user's proximity to a particular sensor or device that has generated the obtained ambient and direct data within the multi-tenant environment. For instance, as a user moves within a particular room in the multi-tenant environment, a sensor or device within the multi-tenant environment may track the user's proximity to the sensor or device and record this proximity as a relative coordinate distance from the sensor or device. The data collection module 104 may use this relative coordinate distance, as well as a known location of the sensor or device, to determine the user's proximity to the sensor or device and extrapolate this information to determine the user's location within the multi-tenant environment.

The data collection module 104, in an embodiment, further processes the obtained ambient and direct data from the different sensors and devices within the multi-tenant environment to identify a set of activity attributes corresponding to a detected user. For instance, the data collection module 104 may process the ambient and direct data through one or more machine learning algorithms or artificial intelligence to detect any activities that a user may be engaged in. The one or more machine learning algorithms or artificial intelligence models may be dynamically trained using supervised, unsupervised, or hybrid training techniques. For instance, a dataset of sample ambient and direct data (e.g., historical ambient and direct data, hypothetical or artificial ambient and direct data, combinations of historical and hypothetical/ambient and direct data, etc.) and sample activities (e.g., known activities associated with the historical ambient and direct data, hypothetical/artificial activities associated with the hypothetical/artificial ambient and direct data, combinations of known and hypothetical/artificial activities, etc.) may be analyzed using a clustering or classification algorithm to classify the sample ambient and direct data according to a set of different classifications (e.g., activities). For instance, the machine learning algorithm may be dynamically trained in real-time by converting the sample ambient and direct data into a set of embeddings and the sample activities into a set of activity embeddings. The machine learning algorithm or artificial intelligence model may classify the sample ambient and direct data according to one or more vectors of similarity between the set of embeddings and the set of activity embeddings. In some instances, the machine learning algorithm or artificial intelligence model may be implemented with a threshold that may be used to determine, based on the aforementioned embeddings and the one or more vectors of similarity, whether sample ambient and direct data is sufficiently similar (e.g., satisfies the threshold by having a requisite amount of embeddings being within a level of tolerance of corresponding activity embeddings for a particular known activity) to a particular sample activity. In some instances, the machine learning algorithm or artificial intelligence model is further trained to detect the absence of ambient and direct data, which may be indicative of an event. For instance, the dataset of sample ambient and direct data may further include null or synthetic ambient and direct data points that may correspond to particular events that represent deviations from routine activities and movements within a sample multi-tenant environment.

In an embodiment, the machine learning algorithm or artificial intelligence model implemented by the data collection module 104 is further trained using simulated and/or test ambient and direct data. The simulated and/or test ambient and direct data used to dynamically train or fine-tune the machine learning algorithm or artificial intelligence model may include hypothetical or artificial ambient and direct data corresponding to different devices and/or sensors that may be implemented in the multi-tenant environment or that are otherwise compatible with the user monitoring gateway 102. In some instances, the simulated and/or test ambient and direct data may include raw sensor and device data from different devices and/or sensors implemented in different testing environments and that are implemented in these different testing environments to generate synthetic, simulated, and/or replay data. This simulated and/or test ambient and direct data may be used to enhance or supplement the dataset used to dynamically train and fine-tune the machine learning algorithm or artificial intelligence implemented by the data collection module 104.

In an embodiment, through the machine learning algorithm or artificial intelligence, the data collection module 104 may perform such clustering or classification of ambient and direct data from different sensors and devices in the multi-tenant environment in real-time or near real-time as the ambient and direct data is received. Through this processing of the ambient and direct data, the data collection module 104 may obtain partial matches among different known activities to detect the activity that a particular user within the multi-tenant environment is engaged in. Example classification and/or clustering algorithms that may be implemented include Support Vector Machines (SVM), k-Nearest Neighbor (KNN) algorithms, logistic regression algorithm, random forest models, Naïve Bayes models, linear regression models, decision tree models, gradient boosting machine models, k-means clustering algorithms, fuzzy c-means (FCM) algorithms, expectation-maximization (EM) algorithms, hierarchical clustering algorithms, density-based spatial clustering of applications with noise (DBSCAN) algorithms and other density-based clustering algorithms (e.g., OPTICS, etc.), balanced iterative reducing and clustering using hierarchies (BIRCH), agglomerative hierarchy (AHC), Gaussian mixture (GMM), spectral clustering algorithms, mini-batch gradient descent algorithms, and the like.

In some instances, the data collection module 104 may combine any identified activities with corresponding timestamps that may be used to further narrow down unique activities performed within the multi-tenant environment. As an illustrative example, if a detected user eats breakfast within a certain time range, the activity attribute corresponding to this activity (eating breakfast) may be supplemented with the timestamp corresponding to this particular time range. This may serve to further identify the particular user engaged in this activity from other possible users within the multi-tenant environment, as described in greater detail herein. Activity attributes may include, but are not limited to, nutrition-based activities (e.g., eating, snacking, drinking, etc.), hygienic activities (e.g., showering, bathing, personal grooming, etc.), resting activities (e.g., sleeping, napping, lying down, etc.), exercise-based activities (e.g., lifting weights, running on a treadmill, using a stationary bicycle, stretching, yoga, etc.), entertainment activities (e.g., watching television, listening to music, playing a video game, etc.), communication-based activities (e.g., calling someone, texting, conversing with another user, etc.), and the like.

In addition to the aforementioned attributes, the data collection module 104 may process the ambient and direct data in real-time or near real-time to generate timing or pacing data that may be aggregated over a period of time for each detected user and according to a corresponding action being performed by the detected user. For example, if the data collection module 104 determines, based on the ambient and direct data, that a particular user is moving within the multi-tenant environment, the data collection module 104 may track the ambient and direct data corresponding to the particular user's movements and dynamically calculate the pace in which the particular user is moving within the multi-tenant environment. As another illustrative example, if the data collection module 104 detects, based on obtained ambient and direct data, that a particular user is present within a particular room of the multi-tenant environment, subsequent ambient and direct data received within a short period of time (e.g., a few seconds, etc.) corresponding to the presence of a user within the particular room may be automatically deemed as being associated with the same particular user.

In an embodiment, the data collection module 104 evaluates the ambient and direct data to automatically detect a particular event occurring within the multi-tenant environment. As noted above, the data collection module 104 may implement and dynamically train a machine learning algorithm or artificial intelligence model to detect any activities that a user may be engaged in. These activities may correspond to observed events occurring within the multi-tenant environment. In some instances, the observed events may be routine for one or more users 110-1-110-N within the multi-tenant environment. As an illustrative example, user 110-1 may be known to routinely eat breakfast between 8:00 A.M. and 8:45 A.M. each day. During this same time range, user 110-2 may be known to routinely move from their bedroom within the multi-tenant environment to a bathroom for personal hygiene and to get ready to start their day. Thus, the likelihood of these detected events corresponding to these particular users may be greater. However, the data collection module 104 may further detect, from the ambient and direct data, particular events that may not be related to a routine activity within the multi-tenant environment. For example, through the aforementioned machine learning algorithm or artificial intelligence, the data collection module 104 may detect an event corresponding to a possible incident within the multi-tenant environment that is not routine, such as an accident within the multi-tenant environment. Further, through the aforementioned machine learning algorithm or artificial intelligence, the data collection module 104 may detect the absence of ambient and direct data corresponding to different activities within the multi-tenant environment. As an illustrative example, if ambient and direct data corresponding to a particular room within the multi-tenant environment is not representative of the presence of a user and/or of an activity occurring within the room, the data collection module 104 may generate attribute data corresponding to the lack of activity within the room. This lack of activity, in some instances, may be indicative of a deviation from routine activities and movements within the multi-tenant environment and, thus, may be indicative of an issue with one or more users within the multi-tenant environment.

In an embodiment, the data collection module 104 aggregates the attribute data according to the detected event within the multi-tenant environment and transmits the aggregated data to a vector computation system 106 implemented by the user monitoring gateway 102. The vector computation system 106 may be implemented as an application or process executing on the user monitoring gateway 102. In some instances, the vector computation system 106 may be implemented through various special-purpose components of the user monitoring gateway 102 that can facilitate real-time or near real-time processing of aggregated attribute data corresponding to different events to generate a set of attribute vectors corresponding to the different events as the aggregated attribute data is received.

In an embodiment, the vector computation system 106 implements and dynamically trains a machine learning algorithm or artificial intelligence model to convert attribute data from the data collection module 104 into a set of embeddings or vector values corresponding to a set of similarity vectors. The machine learning algorithm or artificial intelligence model may be dynamically trained using supervised, unsupervised, or hybrid training techniques. For instance, a dataset of sample attribute data (e.g., known or historical attribute data, hypothetical attribute data, combinations of known/historical and hypothetical attribute data, etc.) and sample vector values corresponding to the sample attribute data (e.g., known vector values corresponding to the different similarity vectors, hypothetical vector values corresponding to the different similarity vectors, combinations of known and hypothetical vector values, etc.) may be analyzed using the machine learning algorithm or artificial intelligence model to convert the sample attribute data into a set of output attribute vector values along the different attribute similarity vectors. This set of output attribute vector values may be compared to the sample vector values in the dataset, which may be the expected vector values for the given input sample attribute data. If any deviations from the sample vector values are identified, the machine learning algorithm or artificial intelligence model may be dynamically retrained or otherwise updated to improve the likelihood of the machine learning algorithm or artificial intelligence model producing the expected vector values for a given attribute data input.

In an embodiment, to dynamically train the machine learning algorithm or artificial intelligence model used to convert attribute data into a set of attribute vectors, the vector computation system 106 generates an initial iteration of the machine learning algorithm or artificial intelligence model. For instance, the vector computation system 106 may initialize a set of coefficients {α1, α2, α3, . . . αn} randomly according to a Gaussian distribution or non-Gaussian distribution. Using this initial iteration of the machine learning algorithm or artificial intelligence, the vector computation system 106 may process the dataset of sample attribute data to generate an output. This output may specify, for each data point corresponding to a particular set of attributes, a set of attribute vector values along different attribute vectors. The vector computation system 106 may compare the output generated using the initial iteration of the machine learning algorithm or artificial intelligence model to the sample vector values defined in the dataset for each of the data points (e.g., sample attribute data) to identify any inaccuracies or other errors.

If the output of the machine learning algorithm or artificial intelligence model does not satisfy one or more criteria, the vector computation system 106 may iteratively update one or more coefficients of the set of coefficients to generate an updated machine learning algorithm or artificial intelligence model. For instance, the one or more criteria may include a threshold for the accuracy of the desired machine learning algorithm or artificial intelligence model for converting attribute data into corresponding attribute vector values. The updated machine learning algorithm or artificial intelligence model may be used to process the aforementioned training dataset, as well as any additional data points or other datasets obtained by the user monitoring gateway 102 to generate a new output for each data point in the training dataset. In some instances, the vector computation system 106 may use an optimization algorithm to iteratively update the one or more coefficients of the set of coefficients associated with the machine learning algorithm or artificial intelligence model. The vector computation system 106 may use this updated machine learning algorithm or artificial intelligence model to process the available data points and generate a new output. The vector computation system 106 may evaluate this new output to determine whether the output satisfies the one or more criteria. This process of updating the set of coefficients associated with the machine learning algorithm or artificial intelligence model according to the one or more criteria may be performed iteratively until an updated machine learning algorithm or artificial intelligence model is produced that satisfies the one or more criteria.

In an embodiment, once the vector computation system 106 has converted the received attribute data into a set of attribute vector values along different attribute similarity vectors, the vector computation system 106 transmits the set of attribute vector values to a user identification engine 108 implemented by the user monitoring gateway 102. The user identification engine 108 may be implemented as an application or process executing on the user monitoring gateway 102. In some instances, the user identification engine 108 may be implemented through various special-purpose components of the user monitoring gateway 102 that can facilitate real-time or near real-time processing of attribute vector values corresponding to different events to identify users associated with these different events as the attribute vector values are received.

In an embodiment, the user identification engine 108 maintains a datastore of known user attributes corresponding to different users known to be associated with the multi-tenant environment. These known user attributes may be generated during an onboarding process. For example, when a user enrolls in a user monitoring service associated with the user monitoring gateway 102, the user may be prompted to provide demographic and other identifying information associated with the user. This demographic and other identifying information may include the user's height, weight, hair color, eye color, age, and the like. In some instances, the user may be prompted to provide images and/or audio recordings of the user that may be used to extract biometric information associated with the user. For example, if the user provides one or more images of the user during the onboarding process, the user monitoring service may process these one or more images using computer vision or other machine learning techniques to extract the user's facial attributes (e.g., facial structure and shape, dental structure, eye color, hair color, facial hair, etc.) and bodily attributes (e.g., body structure and type, skin color, hair color, etc.). As another illustrative example, if the user submits an audial recording of the user, the user monitoring service may process the audial recording using NLP or other machine learning techniques to dynamically generate an audial signature of the user. Using this data corresponding to the user, the user monitoring service may dynamically generate an initial set of attribute vector values corresponding to the user. This initial set of attribute vector values may be stored in the datastore maintained by the user identification engine 108.

In an embodiment, the user identification engine 108 automatically compares the received attribute vector values corresponding to a particular event to the attribute vector values stored in the known user attributes datastore and corresponding to the different users 110-1-110-N within the multi-tenant environment. For instance, the user identification engine 108 may automatically calculate the differences in attribute vector values along each attribute vector to generate a set of value differences (e.g., vector delta values or “deltas”) for each user 110-1-110-N. Based on these vector deltas, the user identification engine 108 may dynamically calculate a user match probability score for each user 110-1-110-N within the multi-tenant environment. In some instances, the user match probability score for a particular user may be calculated by determining the average vector delta between the received attribute vector values and the known attribute vector values associated with the particular user. Alternatively, in some instances, the user match probability score for the particular user may be calculated by calculating the n-dimensional distance between the received attribute vector and the known attribute vector associated with the particular user. Thus, the user match probability score for a particular user may be calculated according to the proximity of the received attribute vector to the known attribute vector corresponding to the particular user, where a match probability score represents a greater likelihood of a match as the level of proximity increases.

Based on these user match probability scores, the user identification engine 108 may identify a user from the users 110-1-110-N that is likely associated with the detected event. For instance, the user identification engine 108 may identify a particular user as being associated with the observed event as a result of the user match probability score corresponding to the particular user and the obtained attribute vector being greater than other user match probability scores corresponding to other users. In an embodiment, prior to associating the ambient and direct data corresponding to the detected event to the identified user, the user identification engine 108 determines whether the calculated user match probability score corresponding to the particular user satisfies a minimum match probability score threshold. This threshold may represent a minimum probability required before an event may be associated with a particular user. Thus, if the calculated user match probability score does not satisfy this threshold, the user identification engine 108 may determine that it cannot associate the detected event with the identified user. Accordingly, the user identification engine 108 may indicate, for the detected event, that it is associated with an unknown user. Alternatively, if the calculated user match probability score satisfies the minimum match probability score threshold, the user identification engine 108 may associate the detected event and corresponding data with the identified user.

By calculating user match probability scores for each user 110-1-110-N and in response to a detected event, the user identification engine 108 reduces the likelihood of the detected event being erroneously associated with a user that is actually not associated with the detected event. As each user 110-1-110-N within the multi-tenant environment may have different notification preferences and thresholds based on the type of event detected, being able to accurately associate a detected event with the correct user is of paramount importance. For instance, if a detected event is associated with a set of obtained biometric readings, these biometric readings may be abnormal for a particular user but otherwise may be within normal ranges for other users within the multi-tenant environment. Thus, if this detected event is erroneously associated with any of these other users, the particular user may not be treated for the possible abnormality. Thus, the user identification engine 108, through processing of attribute vectors obtained from the vector computation system 106, may ensure that detected events are more likely to be associated with the correct users within the multi-tenant environment, ensuring that any abnormalities associated with these users are appropriately addressed.

The user monitoring gateway 102 may automatically transmit this annotated event data 120 to one or more entities according to any applicable user preferences or to the configuration of the user monitoring gateway 102. For instance, if the user monitoring gateway 102 maintains, for each user 110-1-110-N in the multi-tenant environment, a set of notification preferences for delivering event data associated with the user, the user monitoring gateway 102 may refer to the set of notification preferences to select the intended one or more recipients of the event data 120. In some instances, the user monitoring gateway 102 may automatically transmit the event data 120 to the aforementioned user monitoring service, which may process the event data 120 and determine how to route the event data 120 to different intended recipients.

In an embodiment, the user monitoring gateway 102 updates the known attribute vectors corresponding to the users 110-1-110-N within the multi-tenant environment based on feedback from different entities, including the user monitoring service. For example, if an administrator of the user monitoring service assigns a particular event to a particular user (such as in response to feedback from the user, a caregiver, a medical professional, etc.), the user monitoring gateway 102 may evaluate the attribute vector corresponding to the particular event and the known attribute vector corresponding to the identified user to determine which vector values may be updated to incorporate attributes indicated through the attribute vector associated with the particular event. As another illustrative example, if the detected event is confirmed to be associated with the user identified by the user monitoring gateway 102, the user monitoring gateway 102 may use the attribute vector calculated for the detected event to supplement the known attribute vector associated with the identified user to improve the match probability of similar attribute vectors associated with different events to the user.

In an embodiment, the user monitoring gateway 102 further updates the myriad machine learning algorithms or artificial intelligence used to classify received ambient and direct data and to generate different attribute vectors from different attribute data. For example, if the vector computation system 106 calculates a set of attribute vector values that cause the user identification engine 108 to assign the corresponding event to an incorrect user within the multi-tenant environment (e.g., an unknown user or a user other than the actual user associated with the event, etc.), the user monitoring gateway 102 may update the dataset used to train the machine learning algorithm or artificial intelligence model implemented by the vector computation system 106 to add a data point that indicates, for the particular ambient and direct data corresponding to the event, an updated vector that better correlates the detected event to the correct user. This updated vector may be generated by the user monitoring service or other evaluating entity (e.g., administrator, caregiver, medical professional, etc.) according to the known attributes associated with the user and the detected event. Thus, as detected events are associated with different users in real-time or near real-time, the various machine learning algorithms or artificial intelligence used to classify received ambient and direct data and to generate different attribute vectors from different attribute data are continuously updated to better disambiguate ambient and direct data and correctly assign any detected events to the appropriate user within the multi-tenant environment.

It should be noted that the user monitoring gateway 102, through the aforementioned components and corresponding machine learning algorithms or artificial intelligence, can continuously and simultaneously process ambient and direct data from the myriad sensors and devices within the multi-tenant environment to detect, in real-time or near real-time, any events and associate these events with the appropriate users within the multi-tenant environment. For instance, as different ambient and direct data associated with different devices and/or sensors within different portions of the multi-tenant environment is obtained, the user monitoring gateway 102, through the aforementioned components and corresponding machine learning algorithms or artificial intelligence, may simultaneously process this ambient and direct data from these different devices and/or sensors to detect any number of simultaneously occurring events within the multi-tenant environment and assign these simultaneously occurring events to different users within the multi-tenant environment according to the attribute vectors associated with these events and the known attribute vectors corresponding to these different users.

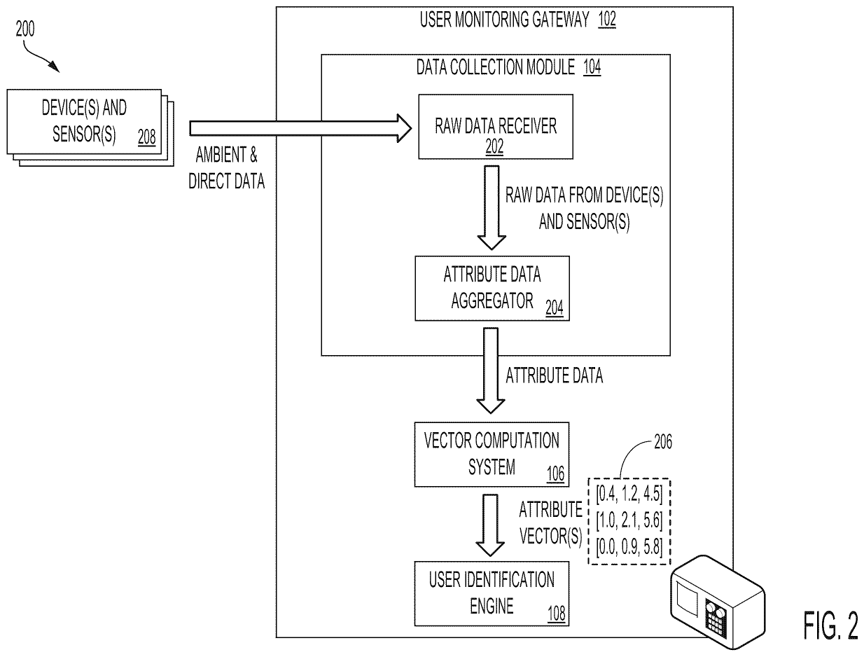

FIG. 2 shows an illustrative example of an environment 200 in which a user monitoring gateway 102 aggregates attribute data for generation of one or more attribute vectors 206 for identification of different users within a multi-tenant environment in accordance with at least one embodiment. As noted above, the user monitoring gateway 102 may implement a data collection module 104 that continuously, and in real-time or near real-time, obtains ambient and direct data associated with the multi-tenant environment from various devices and sensors 208 implemented in the multi-tenant environment and associated with different users within the multi-tenant environment. In the environment 200, the data collection module 104 includes a raw data receiver 202, which may be configured to automatically, and continuously, receive ambient and direct data from the various devices and sensors 208 in real-time or near real-time as the ambient and direct data is generated by these various devices and sensors 208. The raw data receiver 202 may be implemented as an application or sub-process of the data collection module 104 and executing on the user monitoring gateway 102. In some instances, the raw data receiver 202 may be implemented through various special-purpose components of the user monitoring gateway 102 that can facilitate real-time or near real-time processing of ambient and direct data from the various devices and sensors 208 within the multi-tenant environment as this ambient and direct data is generated and transmitted by the various devices and sensors 208. The devices and sensors 208 may include devices and sensors implemented within the multi-tenant environment (e.g., IoT devices 114, sensors 116, cameras 118, and other ambient devices as illustrated in FIG. 1) and personal devices that may be unique to different users within the multi-tenant environment (e.g., personal devices 112-1-112-N as illustrated in FIG. 1) and as described above.

The raw data receiver 202, in an embodiment, implements a set of libraries or other resources corresponding to different data formats utilized by different sensors and devices compatible with the user monitoring gateway 102. As noted above, the ambient and direct data generated and transmitted by the myriad devices and sensors 208 within the multi-tenant environment and connected to the user monitoring gateway 102 through different communications channels may be constructed using different data structures. The raw data receiver 202 may maintain a set of libraries or other resources that indicate these different data structures according to the types of devices and sensors 208 from which the user monitoring gateway 102 may be connected to. Additionally, the raw data receiver 202 may implement a set of libraries or other resources for processing semi-structured and unstructured data from different sensors and devices within the multi-tenant environment.

In an embodiment, the raw data receiver 202 implements computer vision, NLP, and/or other machine learning techniques to process the ambient and direct data from the myriad devices and sensors 208. For example, the raw data receiver 202 may process audiovisual data from the devices and sensors 208 within the multi-tenant environment using computer vision and/or NLP to automatically identify various characteristics associated with the audiovisual data (e.g., user(s) within a room, voice signatures, etc.). As another illustrative example, the raw data receiver 202 may receive telemetry data or other IoT output from one or more IoT devices within the multi-tenant environment that is unstructured and may necessitate format-specific algorithms or other processes for evaluating the telemetry data or other IoT output. Accordingly, the raw data receiver 202 may process this IoT output through these format-specific algorithms or other processes to extract the relevant data usable to identify any attributes corresponding to different events occurring within the multi-tenant environment and corresponding to one or more users within the multi-tenant environment. In some instances, the devices and sensors 208 may be associated with specific locations within the multi-tenant environment (e.g., specific rooms, bathrooms, etc.). Accordingly, the raw data receiver 202 may supplement or enrich ambient and direct data corresponding to different devices and sensors 208 with known location information corresponding to these devices and sensors 208. This known location information may be used, for instance, to denote the location of a user within the multi-tenant environment if the obtained ambient and direct data from a corresponding device or sensor is associated with the user.

In an embodiment, the raw data receiver 202 implements and dynamically trains a machine learning algorithm or artificial intelligence model to process incoming ambient and direct data to identify the format of the ambient and direct data and, accordingly, identify the appropriate library and/or algorithms required to process the obtained ambient and direct data. This machine learning algorithm or artificial intelligence model may be dynamically trained using supervised, unsupervised, or hybrid training techniques. For instance, a dataset of sample ambient and direct data (e.g., known or historical ambient and direct data, hypothetical or artificial ambient and direct data, combinations of known/historical and hypothetical/artificial ambient and direct data, etc.) and sample libraries and/or algorithms (e.g., known libraries or algorithms usable to process the sample ambient and direct data, etc.) may be analyzed using a clustering or classification algorithm to classify the sample ambient and direct data according to a set of different classifications (i.e., libraries and/or algorithms for processing ambient and direct data). For instance, the machine learning algorithm or artificial intelligence model may be dynamically trained in real-time by converting the sample ambient and direct data into a set of ambient and direct data embeddings and sample data known to be compatible with the different libraries and/or algorithms into a set of format embeddings. The machine learning algorithm or artificial intelligence model may classify the sample ambient and direct data according to one or more vectors of similarity between the set of ambient and direct data embeddings and the set of format embeddings. Example classification and/or clustering algorithms that may be implemented include Support Vector Machines (SVM), k-Nearest Neighbor (KNN) algorithms, logistic regression algorithm, random forest models, Naïve Bayes models, linear regression models, decision tree models, gradient boosting machine models, and the like.

The machine learning algorithm or artificial intelligence model implemented by the raw data receiver 202 may be evaluated to determine whether the machine learning algorithm or artificial intelligence model is identifying the correct libraries and/or algorithms for processing the ambient and direct data. For instance, using the aforementioned dataset as input, an evaluator of the machine learning algorithm or artificial intelligence model may determine whether the output generated by the machine learning algorithm or artificial intelligence model (i.e., selected libraries and/or algorithms) correspond to the input sample ambient and direct data. If the machine learning algorithm or artificial intelligence model is producing erroneous classifications of the sample ambient and direct data (e.g., the machine learning algorithm or artificial intelligence model selects incorrect libraries and/or algorithms for processing the sample ambient and direct data, etc.), the machine learning algorithm or artificial intelligence model may be updated to increase the likelihood of the machine learning algorithm or artificial intelligence model producing the correct classifications of the sample ambient and direct data. For instance, the raw data receiver 202 may iteratively update one or more coefficients of a set of coefficients associated with the machine learning algorithm or artificial intelligence model to generate an updated machine learning algorithm or artificial intelligence model. The training dataset described above may be processed using this updated machine learning algorithm or artificial intelligence model to generate a new classifications of the ambient and direct data that may be evaluated to determine whether the machine learning algorithm or artificial intelligence model is producing the correct or expected classifications of the ambient and direct data. This iterative process may continue until an iteration of the machine learning algorithm or artificial intelligence model is produced that satisfies one or more criteria (e.g., accuracy threshold, etc.) for identifying the appropriate libraries and/or algorithms for extracting attribute data from the ambient and direct data.

In an embodiment, the machine learning algorithm or artificial intelligence model implemented by the raw data receiver 202 is further trained using simulated and/or test ambient and direct data. The simulated and/or test ambient and direct data used to dynamically train or fine-tune the machine learning algorithm or artificial intelligence model may include hypothetical or artificial ambient and direct data corresponding to different devices and/or sensors that may be implemented in the multi-tenant environment or that are otherwise compatible with the user monitoring gateway 102. In some instances, the simulated and/or test ambient and direct data may include raw sensor and device data from different devices and/or sensors implemented in different testing environments and that are implemented in these different testing environments to generate synthetic, simulated, and/or replay data. This simulated and/or test ambient and direct data may be used to enhance or supplement the dataset used to dynamically train and fine-tune the machine learning algorithm or artificial intelligence implemented by the raw data receiver 202.

In an embodiment, the raw data receiver 202 transmits the raw data from the different devices and sensors 208 in the multi-tenant environment, as well as the identified libraries and/or algorithms for processing the raw data, to an attribute data aggregator 204 implemented by the data collection module 104. The attribute data aggregator 204 may be implemented as an application or sub-process of the data collection module 104 and executing on the user monitoring gateway 102. In some instances, the attribute data aggregator 204 may be implemented through various special-purpose components of the user monitoring gateway 102 that can facilitate real-time or near real-time processing of raw data from the raw data receiver 202 (i.e., the ambient and direct data from the various devices and sensors 208 within the multi-tenant environment) as this ambient and direct data is generated and transmitted by the various devices and sensors 208.

The attribute data aggregator 204, in an embodiment, processes the received raw data from the raw data receiver 202 using the identified libraries and/or algorithms associated with the received raw data to extract attribute data corresponding to different users and events detected within the multi-tenant environment. As noted above, ambient and direct data collected from the various devices and sensors 208 within the multi-tenant environment may be processed to detect physical attributes associated with any user detected within the multi-tenant environment, location attributes corresponding to any user detected within the multi-tenant environment, activity attributes corresponding to any events detected within the multi-tenant environment, and timing/pacing attributes corresponding to any detected users and to the events detected within the multi-tenant environment. The attribute data aggregator 204 may process the raw data provided by the raw data receiver 202 through the identified libraries and/or algorithms associated with the received raw data to aggregate the ambient and direct data in a consistent format usable by the attribute data aggregator 204 to extract any attribute data corresponding to the aforementioned attribute data categories.

In an embodiment, the attribute data aggregator 204 implements one or more machine learning algorithms or artificial intelligence that are dynamically trained to extract and aggregate attribute data from the obtained raw data according to different attribute data categories. For example, if the raw data includes audiovisual data, the attribute data aggregator 204 may process this audiovisual data using computer vision and/or NLP to obtain data corresponding to the physical attributes of any users detected within the multi-tenant environment and associated with the detected events. As another illustrative example, using computer vision, the attribute data aggregator 204 may dynamically evaluate any images and/or video recordings of the multi-tenant environment to calculate a user's location within the multi-tenant environment relative to any of the devices and sensors 208 that collected the ambient and direct data within the multi-tenant environment. In yet another illustrative example, the attribute data aggregator 204 may implement one or more machine learning algorithms or artificial intelligence to detect, from the raw data, any activities that a user may be engaged in.

The attribute data aggregator 204 may associate the attribute data extracted from the obtained raw data according to any events detected within the multi-tenant environment. Returning to an earlier illustrative example, the data collection module 104, through the attribute data aggregator 204, may implement and dynamically train a machine learning algorithm or artificial intelligence model to detect any activities that a user may be engaged in. These activities may correspond to observed events occurring within the multi-tenant environment. Thus, these observed events may be routine for one or more users within the multi-tenant environment. The attribute data aggregator 204, in addition to these routine events, may detect events that may not be related to a routine activity within the multi-tenant environment. For example, through the aforementioned machine learning algorithm or artificial intelligence, the attribute data aggregator 204 may detect an event corresponding to a possible incident within the multi-tenant environment that is not routine, such as an accident within the multi-tenant environment. Further, through the aforementioned machine learning algorithm or artificial intelligence, the attribute data aggregator 204 may detect the absence of ambient and direct data corresponding to different activities within the multi-tenant environment. As an illustrative example, if ambient and direct data corresponding to a particular room within the multi-tenant environment is not representative of the presence of a user and/or of an activity occurring within the room, the attribute data aggregator 204 may generate attribute data corresponding to the lack of activity within the room. This lack of activity, in some instances, may be indicative of a deviation from routine activities and movements within the multi-tenant environment and, thus, may be indicative of an issue with one or more users within the multi-tenant environment.

The attribute data aggregator 204, in some instances, may aggregate the attribute data extracted from the raw data obtained from the raw data receiver 202 according to the corresponding detected event within the multi-tenant environment. For instance, the attribute data aggregator 204 may annotate the attribute data according to each attribute category and to the particular detected event identified as being associated with the attribute data. Thus, for a given detected event, the attribute data aggregator 204 may include the physical attributes corresponding to any associated users, location attributes corresponding to these associated users and the detected event, activity attributes corresponding to the detected event, and the timing/pacing attributes corresponding to the associated users and the occurrence of the detected event. Once the attribute data aggregator 204 has aggregated the attribute data for each detected event, the attribute data aggregator 204 may transmit the aggregated attribute data to the vector computation system 106.

As noted above, the vector computation system 106 may implement and dynamically train a machine learning algorithm or artificial intelligence model to convert attribute data from the attribute data aggregator 204 into a set of embeddings or vector values corresponding to a set of similarity vectors. This machine learning algorithm or artificial intelligence model may be dynamically trained using a dataset of sample attribute data and sample vector values corresponding to the sample attribute data, which may be analyzed using the machine learning algorithm or artificial intelligence model to convert the sample attribute data into a set of output attribute vector values along the different attribute similarity vectors. This set of output attribute vector values may be compared to the sample vector values in the dataset such that the machine learning algorithm or artificial intelligence model may be dynamically updated according to any deviations from the sample vector values. The vector computation system 106 may process the attribute data from the attribute data aggregator 204 through this machine learning algorithm or artificial intelligence model to generate a corresponding attribute vector 206 having vector values corresponding to the different attributes indicated in the attribute data.

The vector computation system 106 may transmit the newly calculated attribute vector 206 to the user identification engine 108 to identify a particular user that may be associated with the corresponding event for which the attribute vector 206 was calculated. As noted above, the user identification engine 108 may maintain a datastore of known user attributes corresponding to different users known to be associated with the multi-tenant environment. The user identification engine 108 may automatically compare the received attribute vector 206 corresponding to a particular event to the attribute vectors stored in the known user attributes datastore and corresponding to the different users within the multi-tenant environment. For instance, the user identification engine 108 may automatically calculate the differences in attribute vector values along each attribute vector dimension to generate a set of value differences (e.g., vector deltas) for each user. Based on these vector deltas, the user identification engine 108 may dynamically calculate a user match probability score for each user within the multi-tenant environment. In some instances, the user match probability score for a particular user may be calculated by determining the average vector delta between the received attribute vector values and the known attribute vector values associated with the particular user. Alternatively, in some instances, the user match probability score for the particular user may be calculated by determining the n-dimensional distance between the received attribute vector 206 and the known attribute vector associated with the particular user. Thus, the user match probability score for a particular user may be calculated according to the proximity of the received attribute vector to the known attribute vector corresponding to the particular user, where a match probability score represents a greater likelihood of a match as the level of proximity increases. Based on these user match probability scores, the user identification engine 108 may identify a user that is likely associated with the detected event.

As noted above, by calculating user match probability scores for each known user within the multi-tenant environment and in response to a detected event, the user identification engine 108 reduces the likelihood of the detected event being erroneously associated with a user that is actually not associated with the detected event. As each user within the multi-tenant environment may have different notification preferences and thresholds based on the type of event detected, being able to accurately associate a detected event with the correct user is of paramount importance. Returning to an earlier illustrative example, if a detected event is associated with a set of obtained biometric readings, these biometric readings may be abnormal for a particular user but otherwise may be within normal ranges for other users within the multi-tenant environment. Thus, if this detected event is erroneously associated with any of these other users, the particular user that is actually associated with these biometric readings may not be treated for the possible abnormality. Thus, the user identification engine 108, through processing of attribute vectors 206 obtained from the vector computation system 106, may ensure that detected events are more likely to be associated with the correct users within the multi-tenant environment, ensuring that any abnormalities associated with these users are appropriately addressed.

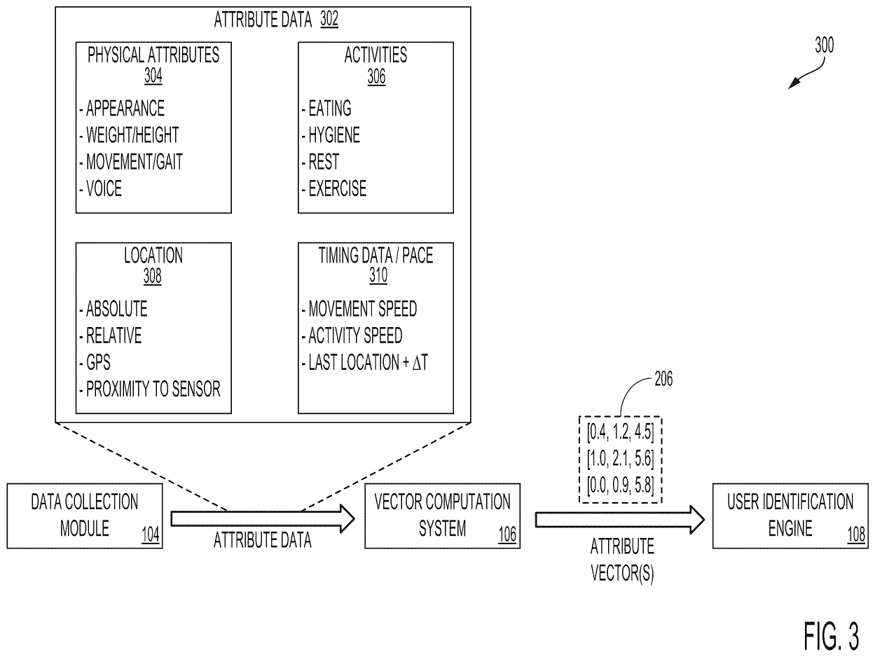

FIG. 3 shows an illustrative example of an environment 300 in which a data collection module 104 implemented by a user monitoring gateway aggregates attribute data 302 according to different attribute categories 304-310 for calculation of attribute vectors 206 and identification of users within a multi-tenant environment in accordance with at least one embodiment. In the environment 300, the data collection module 104 may process ambient and direct data collected through various sensors and devices implemented in a multi-tenant environment to extract attribute data 302 that may be used to associate detected events with corresponding users within the multi-tenant environment. As noted above, the attribute data 302 may include physical attribute data 304 corresponding to the physical attributes of any users detected within the multi-tenant environment that may be associated with a particular event. These physical attributes may include, for instance, the physical appearance of a detected user such as the detected user's facial and bodily attributes, the detected user's weight and height, the detected user's movement or gait within the multi-tenant environment, the detected user's voice signature, and the like.

In an embodiment, the data collection module 104 identifies the physical attribute data 304 through processing of the ambient and direct data through one or more machine learning algorithms or artificial intelligence. For example, the data collection module 104 may process the ambient and direct data using computer vision, NLP, or other machine learning technique to evaluate any included audiovisual data and identify an approximate height and weight associated with any detected user. Additionally, by processing this audiovisual data using computer vision or other machine learning algorithm, the data collection module 104 may collect various data points corresponding to a detected user's position within a room that may be aggregated to define the detected user's movement and gait within the room. As another illustrative example, the data collection module 104 may process any audial data using NLP or other voice-recognition machine learning algorithm to generate a voice signature of any detected user within the multi-tenant environment. The voice signature may correspond to different characteristics associated with the user's voice that may be used to uniquely identify the user based on their audial communications.

The attribute data 302 generated by the data collection module 104 may further include activity data 306 corresponding to different activities that may be occurring within the multi-tenant environment and that may be detected by the myriad sensors and devices within the multi-tenant environment. As noted above, the data collection module 104 may implement and dynamically train one or more machine learning algorithms or artificial intelligence to process obtained ambient and direct data to detect any activities that may be occurring within the multi-tenant environment. These one or more machine learning algorithms or artificial intelligence may include a clustering or classification algorithm that may classify received ambient and direct data according to a set of different classifications (e.g., activities) by converting the ambient and direct data into a set of ambient and direct data embeddings. This set of ambient and direct data embeddings may be compared to different activity embeddings corresponding to different known activities along one or more vectors of similarity to identify the one or more activities that may be occurring within the multi-tenant environment. In some instances, the one or more machine learning algorithms or artificial intelligence may be implemented with a threshold that may be used to determine, based on the aforementioned embeddings and the one or more vectors of similarity, whether the received ambient and direct data is sufficiently similar (e.g., satisfies a classification threshold by having a requisite amount of embeddings being within a level of tolerance of corresponding activity embeddings for a particular known activity) to a particular activity. As noted above, the data collection module 104 may combine any identified activities with corresponding timestamps that may be used to further narrow down unique activities performed within the multi-tenant environment. The activity data 306 may include, but is not limited to, data corresponding to nutrition-based activities (e.g., eating, snacking, drinking, etc.), hygienic activities (e.g., showering, bathing, personal grooming, etc.), resting activities (e.g., sleeping, napping, lying down, etc.), exercise-based activities (e.g., lifting weights, running on a treadmill, using a stationary bicycle, stretching, yoga, etc.), entertainment activities (e.g., watching television, listening to music, playing a video game, etc.), communication-based activities (e.g., calling someone, texting, conversing with another user, etc.), and the like.

The attribute data 302 generated by the data collection module 104 may further include location data 308 corresponding to any location attributes associated with any user detected within the multi-tenant environment. The location data 308 may include, for instance, the absolute location of a detected user within the multi-tenant environment at a given time. As noted above, an absolute location may, for instance, correspond to a particular room or other portion of the multi-tenant environment. In response to receiving ambient and direct data from the different sensors and devices within the multi-tenant environment, the data collection module 104 may process the ambient and direct data to detect which room or other portion of the multi-tenant environment the ambient and direct data originated from. If, through processing of the ambient and direct data, the data collection module 104 detects that a user is associated with the ambient and direct data, the data collection module 104 may associate the detected user with the absolute location from which the corresponding ambient and direct data was obtained. The location data 308 may further include the relative location of a detected user over a period of time. For instance, if a detected user is moving between different rooms within the multi-tenant environment and two distinct sensors and/or devices detect the user within these different rooms over a short period of time, the data collection module 104 may process the ambient and direct data corresponding to these two distinct sensors and/or devices to detect the user's transition from one room to another and, thus, determine the user's relative location within the multi-tenant environment. The location data 308 may further include any geolocation data corresponding to a detected user, such as GPS coordinates obtained from sensors and/or devices that implement a GPS receiver or antenna or from one or more user's personal devices. In some instances, the location data 308 may additionally include location proximity data that represents a detected user's proximity to a particular sensor or device that has generated the obtained ambient and direct data within the multi-tenant environment. For instance, as a user moves within a particular room in the multi-tenant environment, a sensor or device within the multi-tenant environment may track the user's proximity to the sensor or device and record this proximity as a relative coordinate distance from the sensor or device.

As noted above, the data collection module 104 may process the ambient and direct data in real-time or near real-time to generate timing or pacing data 310 that may be aggregated over a period of time for each detected user and according to a corresponding action being performed by the detected user. Returning to an earlier illustrative example, if the data collection module 104 determines, based on the ambient and direct data, that a particular user is moving within the multi-tenant environment, the data collection module 104 may track the ambient and direct data corresponding to the particular user's movements and dynamically calculate the pace at which the particular user is moving within the multi-tenant environment. As another illustrative example, if the data collection module 104 detects, based on obtained ambient and direct data, that a particular user is present within a particular room of the multi-tenant environment, subsequent ambient and direct data received within a short period of time (e.g., a few seconds, etc.) corresponding to the presence of a user within the particular room may be automatically deemed as being associated with the same particular user. Thus, based on this ambient and direct data, the data collection module 104 may dynamically calculate, for a detected user, their movement speed, the speed at which they are engaging in a detected activity (e.g., duration of the detected activity, etc.), their last location and the time interval since they were last detected being in the location, and the like.

The data collection module 104 may aggregate the attribute data 302 according to the aforementioned data categories 304-310 and transmit the aggregated attribute data 302 to the vector computation system 106. As noted above, the vector computation system 106 may process this aggregated attribute data 302 through a machine learning algorithm or artificial intelligence model to generate a corresponding attribute vector 206 having vector values corresponding to the different attributes indicated in the attribute data 302. This attribute vector 206 may be processed by the user identification engine 108, as described above, to identify a user that may be associated with the underlying activity or event occurring within the multi-tenant environment.

In some instances, the attribute data 302 used to generate a corresponding attribute vector 206 may initially be devoid of certain data corresponding to different user attributes. For instance, when a new user is introduced to the multi-tenant environment and is onboarded onto the user monitoring gateway, the new user may provide basic attribute data associated with the new user (e.g., age, height, weight, any known medical conditions or other biometric attributes, etc.). As the new user is detected by different devices and sensors within the multi-tenant environment, or personal devices associated with the new user transmit data to the data collection module 104, the data collection module 104 may continuously aggregate this data, which may result in updated attribute vectors associated with the new user. The user identification engine 108 may process these updated attribute vectors to continuously identify any new user attributes associated with this new user as the new user continues their activities in the multi-tenant environment. Thus, over time, the user identification engine 108 may continuously update the attribute vectors associated with each user within the multi-tenant environment to better reflect the attributes or characteristics of these users.

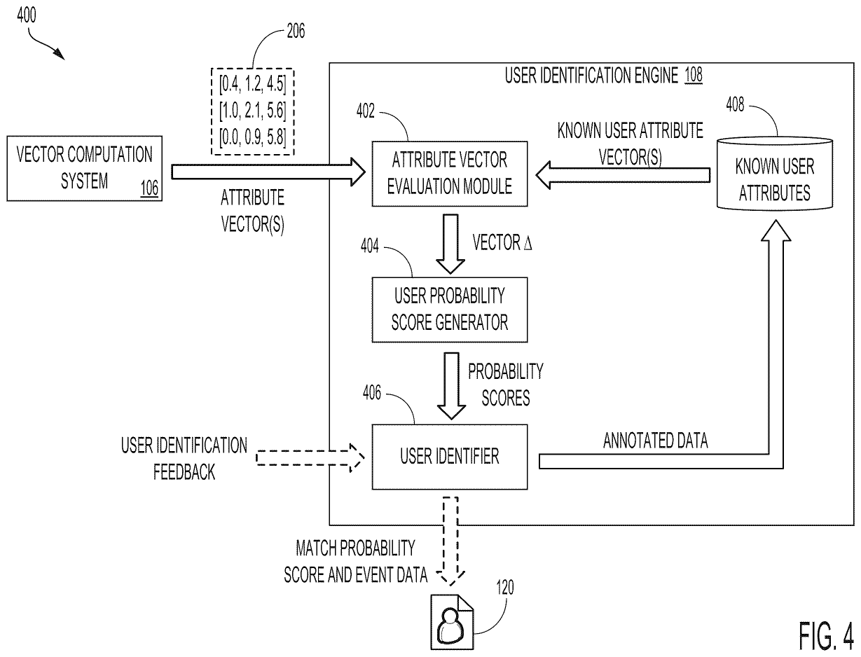

FIG. 4 shows an illustrative example of an environment 400 in which a user identification engine 108 implemented by a user monitoring gateway dynamically processes attribute vectors 206 corresponding to received ambient and direct data within a multi-tenant environment to identify a possible user associated with a detected event in accordance with at least one embodiment. In the environment 400, the vector computation system 106, upon calculating an attribute vector 206 corresponding to a particular event detected by the user monitoring gateway, transmits this attribute vector 206 to the user identification engine 108 to determine whether the particular event can be associated with a known user within the multi-tenant environment. As noted above, the attribute vector 206 may be an n-dimensional vector having values corresponding to different attributes that may be associated with the detected event and a user within the multi-tenant environment.

In an embodiment, the attribute vector 206 generated by the vector computation system 106 may be received by an attribute vector evaluation module 402 implemented by the user identification engine 108. The attribute vector evaluation module 402 may be implemented as an application or sub-process of the user identification engine 108 and executing on the user monitoring gateway. In some instances, the attribute vector evaluation module 402 may be implemented through various special-purpose components of the user monitoring gateway that can facilitate real-time or near real-time processing of attribute vectors corresponding to different detected events within the multi-tenant environment as ambient and direct data corresponding to these events is generated and transmitted by the various devices and sensors to the user monitoring gateway.

In response to receiving an attribute vector 206 corresponding to a detected event, the attribute vector evaluation module 402 may query a known user attributes datastore 408 to retrieve any available attribute vectors corresponding to different users known to be associated with the particular multi-tenant environment. As noted above, the known user attributes associated with these different users may be generated during an onboarding process. For example, when a user enrolls in a user monitoring service associated with the user monitoring gateway, the user may be prompted to provide demographic and other identifying information associated with the user. This demographic and other identifying information may include the user's height, weight, hair color, eye color, age, and the like. In some instances, the user may be prompted to provide images and/or audio recordings of the user that may be used to extract biometric information associated with the user. For example, if the user provides one or more images of the user during the onboarding process, the user monitoring service may process these one or more images using computer vision or other machine learning techniques to extract the user's facial attributes (e.g., facial structure and shape, dental structure, eye color, hair color, facial hair, etc.) and bodily attributes (e.g., body structure and type, skin color, hair color, etc.). As another illustrative example, if the user submits an audial recording of the user, the user monitoring service may process the audial recording using NLP or other machine learning techniques to dynamically generate an audial signature of the user. In some instances, the user may be prompted to indicate what activities the user may engage in within the multi-tenant environment and when these activities commonly occur. Using this data corresponding to the user, the user monitoring service may dynamically generate an initial set of attribute vector values corresponding to the user. This initial set of attribute vector values may be stored in the known user attributes datastore 408.

The attribute vector evaluation module 402 may compare each known user attribute vector to the received attribute vector 206 to compute the value differences (i.e., vector delta or “Δ”) along each dimension of the n-dimensional vector. The length of this resultant vector may be inversely proportional to the level of similarity between the received attribute vector 206 and the known user attribute vector corresponding to a particular user. Thus, the attribute vector evaluation module 402, through evaluation of the attribute vector 206 against the available known user attribute vectors stored within the known user attributes datastore 408, may generate different resultant difference vectors (i.e., vector deltas or “Δ”) for each known user associated with the multi-tenant environment or otherwise associated with the user monitoring gateway. The attribute vector evaluation module 402 may transmit these resultant difference vectors to a user probability score generator 404 to determine the probability of the detected event being associated with each known user.

The user probability score generator 404 may be implemented as an application or sub-process of the user identification engine 108 and executing on the user monitoring gateway. In some instances, the user probability score generator 404 may be implemented through various special-purpose components of the user monitoring gateway that can facilitate real-time or near real-time processing of resultant vectors or vector deltas corresponding to comparisons between attribute vectors corresponding to different detected events within the multi-tenant environment and to known user attribute vectors. This real-time or near real-time processing by the user probability score generator 404 may be performed as ambient and direct data corresponding to these detected events is generated and transmitted by the various devices and sensors to the user monitoring gateway.

In an embodiment, the user probability score generator 404 implements one or more functions that may be applied to received resultant difference vectors or vector deltas to compute a user probability score for each known user associated with the multi-tenant environment or otherwise associated with the user monitoring gateway. As noted above, the length of a resultant difference vector may be inversely proportional to the likelihood of an associated event corresponding to a particular user. For example, a longer resultant difference vector may serve as an indication that there is little similarity between the attribute vector 206 and the evaluated known user attribute vector associated with a particular user. Alternatively, a shorter or null resultant difference vector (e.g., a vector having zero length) may denote a greater level of similarity between the attribute vector 206 and the evaluated known user attribute vector associated with a particular user. The one or more functions implemented by the user probability score generator 404 may thus be defined such that the resulting probability score denotes a higher probability that a detected event is associated with a particular user as the length of the resultant difference vector associated with this particular user decreases.

As the user probability score generator 404 generates user probability scores for each known user associated with the multi-tenant environment and/or the user monitoring gateway, the user probability score generator 404 may transmit these probability scores to a user identifier 406 implemented by the user identification engine 108. The user identifier 406 may be implemented as an application or sub-process of the user identification engine 108 and executing on the user monitoring gateway. In some instances, the user identifier 406 may be implemented through various special-purpose components of the user monitoring gateway that can facilitate real-time or near real-time processing of user probability scores corresponding to different users and for a detected event to determine whether the detected event is likely associated with a particular user within the multi-tenant environment. This real-time or near real-time processing by the user identifier 406 may be performed as ambient and direct data corresponding to these detected events is generated and transmitted by the various devices and sensors to the user monitoring gateway.

In an embodiment, the user identifier 406 implements an identification threshold that is used to determine whether the detected event can be associated with a known user. This identification threshold, for example, may denote a minimum probability score required for a user to be deemed, with a high (acceptable or adequate) degree of certainty, to be associated with the detected event. Accordingly, the user probability score generator 404 may evaluate each calculated probability score provided by the user probability score generator 404 to identify any user probability scores that satisfy this identification threshold. By maintaining an identification threshold for associating a detected event with a known user, the user identifier 406 ensures that a detected event that cannot, with a sufficient degree of certainty, be associated with a known user is maintained as corresponding to an unknown user within the multi-tenant environment. This reduces the likelihood of such detected events being erroneously associated with any known user associated with the multi-tenant environment. Further, this may allow for identification of new users within the multi-tenant environment and for which new known user attribute vectors may be defined. In some instances, if the detected event is later associated with a known user associated with the multi-tenant environment, the attribute vector corresponding to the detected event may be processed to update the known user attribute vector corresponding to the known user. This may allow for the refinement of the known user attribute vector so that the known user attribute vector better reflects the actual characteristics or attributes of the known user.

If the user identifier 406 determines that the user probability score corresponding to the highest probability of a detected event being associated with a particular user satisfies the identification threshold implemented by the user identifier 406, the user identifier 406 may associate the detected event with the particular user. Accordingly, the user identifier 406 may obtain the ambient and direct data associated with the detected event and may annotate this ambient and direct data to indicate that the ambient and direct data is associated with the particular user. This annotated data may be added to an entry corresponding to the particular user in the known user attributes datastore 408. As noted above, the ambient and direct data and the corresponding attribute vector 206 may be used by the user identification engine 108 to enhance or otherwise update the attribute vector corresponding to the particular user.

If the user identifier 406 determines that no user probability score provided by the user probability score generator 404 satisfies the identification threshold (e.g., no user probability score is greater than the required minimum user probability score for identification, etc.), the user identifier 406 may determine that the detected event cannot be associated with a known user within the multi-tenant environment or otherwise associated with the user monitoring gateway. Accordingly, the user identifier 406 may annotate the ambient and direct data associated with the detected event to indicate that the detect event corresponds to an unknown user. In an embodiment, the user identifier 406 updates the known user attributes datastore 408 by introducing a new entry corresponding to this detected event and its association with an unknown user. This new entry may serve as a placeholder for when a particular user is identified as being associated with the detected event and the corresponding attribute vector 206 can be used to generate a known user attribute vector for the particular user.

Once the user identifier 406 has associated a particular event with a known or unknown user, the user identifier 406 may automatically transmit this annotated event data 120 to one or more entities according to any applicable user preferences or to the configuration of the user monitoring gateway. As noted above, if the user monitoring gateway maintains, for each user in the multi-tenant environment, a set of notification preferences for delivering event data associated with the user, the user identifier 406 may refer to the set of notification preferences to select the intended one or more recipients of the event data 120. In some instances, the user identifier 406 may automatically transmit the event data 120 to a remote user monitoring service, which may process the event data 120 and determine how to route the event data 120 to different intended recipients.

In an embodiment, the user identifier 406 continuously, in real-time or near real-time, receives feedback corresponding to the association of different users (known and unknown users) to detected events within the multi-tenant environment within which the user monitoring gateway is implemented. Returning to an earlier illustrative example, if an administrator of the user monitoring service assigns a particular event to a particular user (such as in response to feedback from the user, a caregiver, a medical professional, etc.), the user identifier 406 may evaluate the attribute vector 206 corresponding to the particular event and the known attribute vector corresponding to the identified user to determine whether any of the vector values associated with the known attributed vector may be updated to incorporate attributes indicated through the attribute vector associated with the particular event. As another illustrative example, if the detected event is confirmed to be associated with the user identified by the user identifier 406, the user identifier 406 may use the attribute vector 206 calculated for the detected event to supplement the known attribute vector associated with the identified user to improve the likelihood (e.g., user probability scores) of similar attribute vectors associated with different events being linked to the user.

In some instances, if a detected event was associated with an unknown user by the user identifier 406 and the user identifier 406 receives feedback indicating an identity of a particular user associated with the detected event, the user identifier 406 may update the entry corresponding to this detected event to associate the attribute vector corresponding to this detected event to the particular user. For instance, if the identified user was previously not known to the user monitoring gateway (i.e., there was no entry within the known user attributes datastore 408 corresponding to the identified user), the user identifier 406 may automatically update the newly generated entry corresponding to the detected event to associate the entry with the identified user such that new attribute vectors corresponding to newly detected events may be evaluated by the attribute vector evaluation module 402 against the attribute vector now associated with this identified user to determine whether any of these newly detected events are associated with the identified user as opposed to being classified as being associated with an unknown user.

In an embodiment, if the particular event was previously associated with an unknown user and the user identifier 406 receives feedback indicating that the particular event is associated with a user for which a known user attribute vector is maintained in the known user attributes datastore 408, the user identifier 406 dynamically updates the known user attribute vector associated with the known user to incorporate the attribute vector corresponding to the particular event. For instance, the user identifier 406 may process the known user attribute vector corresponding to the identified user and the attribute vector associated with the particular event through an optimization algorithm to compute the new n-dimensional values for the known user attribute vector, incorporating the detected attributes of the particular event now associated with the identified user.

In some instances, the feedback received by the user identifier 406 may include an updated attribute vector corresponding to the particular event for which the user identifier 406 previously provided event data 120. For instance, if a particular event was previously associated with an unknown or otherwise incorrect user by the user identifier 406, an administrator of the user monitoring gateway or other evaluating entity (e.g., a user, a caregiver, a medical professional, etc.) may dynamically update the attribute vector associated with the particular event to incorporate one or more attributes associated with the correct user associated with the particular event. The user identifier 406 may accordingly use this updated attribute vector to supplement an existing attribute vector associated with this user and maintained in the known user attributes datastore 408, as described above. If the updated attribute vector is associated with a new user not previously known to the user monitoring gateway, the user identifier 406 may generate a new entry in the known user attributes datastore 408 corresponding to this new user and incorporate the updated attribute vector with this new entry.

In an embodiment, the feedback obtained by the user identifier 406 is further used to dynamically update one or more machine learning algorithms or artificial intelligence implemented by the user monitoring gateway. For example, as noted above, the vector computation system 106 implements and dynamically trains a machine learning algorithm or artificial intelligence model to convert attribute data corresponding to a detected event and to a user within the multi-tenant environment into a set of embeddings or vectorial values corresponding to a set of similarity vectors in an n-dimensional space. The dataset used to dynamically train this machine learning algorithm or artificial intelligence model may be updated to incorporate the updated attribute vector corresponding to the particular event and identified user, as well as any annotations indicating how the attributes associated with the event and user should be converted into this particular attribute vector. The machine learning algorithm or artificial intelligence model may process this updated dataset to generate a new output that may be evaluated according to the included annotations to determine whether the machine learning algorithm or artificial intelligence model is accurately converting attribute data into corresponding attribute vectors for event and user identification. Based on this evaluation, the machine learning algorithm or artificial intelligence model may be iteratively updated until a new iteration of the machine learning algorithm or artificial intelligence model is generated that satisfies one or more criteria for generating attribute vectors.

It should be noted that, in some instances, the known user attribute vectors associated with different users in the multi-tenant environment may be updated or re-established periodically or in response to a triggering event in order to maintain a current baseline set of user attributes usable to detect new deviations that may be indicative of events impacting these users. For example, if a user experiences a significant health-related event that results in a change of their baseline attributes, the known user attribute vectors associated with this user may be updated according to their new baseline attributes. As another illustrative example, at pre-defined time intervals, the user may be prompted to indicate new baseline attributes that may be used to define new known user attribute vectors for the user. In some instances, the attribute values included in the known user attribute vectors may be accompanied by different weighting factors that may be used to assign different weight to attribute data obtained from different devices and sensors. For example, if attribute data corresponding to a user's heart rate is obtained from a wearable device and from an embedded pacemaker device associated with the user, the user identification engine 108 may apply greater weight to the attribute data originating from the embedded pacemaker device, as this device may be permanently associated with the user as opposed to a wearable device that may be removed or otherwise used by other users.

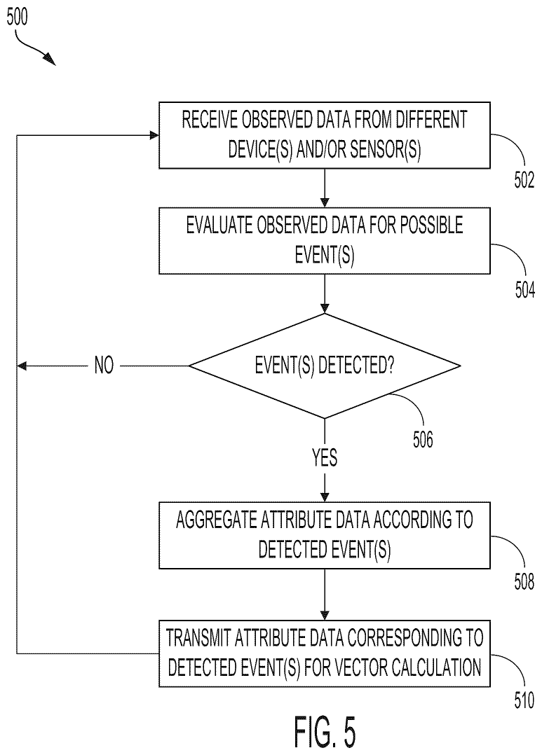

FIG. 5 shows an illustrative example of a process 500 for aggregating attribute data corresponding to one or more possible users within a multi-tenant environment in response to detecting an event within the multi-tenant environment in accordance with at least one embodiment. The process 500 may be performed by the data collection module (e.g., data collection module 104 described above in connection with FIGS. 1-3) implemented by the user monitoring gateway. At step 502, the data collection module may receive observed data from different devices and/or sensors implemented in a multi-tenant environment. As noted above, the user monitoring gateway may maintain different communications channels with different devices and/or sensors implemented within the multi-tenant environment. These different communications channels or connections may be established through different communications protocols, including any IoT protocols, wireless communications protocols, and the like. These different communications channels or connections may be persistent, whereby as the different devices and/or sensors generate data corresponding to different events occurring in the multi-tenant environment, the different devices and/or sensors (through the different communications channels or connections) may transmit this data in real-time or near real-time to the user monitoring gateway. The different communications channels or connections with the different devices and/or sensors within the multi-tenant environment may be facilitated through the data collection module, which may dynamically receive observed data from these different devices and/or sensors as the observed data is generated.

At step 504, the data collection module may evaluate the observed data for possible events occurring within the multi-tenant environment. As noted above, the data collection module may implement and dynamically train a machine learning algorithm or artificial intelligence model to detect any activities that a user may be engaged in within the multi-tenant environment. These activities may correspond to observed events occurring within the multi-tenant environment. In some instances, the observed events may be routine for one or more users within the multi-tenant environment (e.g., eating, sleeping, showering/bathing, exercising, etc.). The data collection module may further detect, from the observed data, particular events that may not be related to a routine activity within the multi-tenant environment. Returning to an earlier illustrative example, through the aforementioned machine learning algorithm or artificial intelligence, the data collection module may detect an event corresponding to a possible incident within the multi-tenant environment that is not routine, such as an accident within the multi-tenant environment. Further, through the aforementioned machine learning algorithm or artificial intelligence, the data collection module may detect the absence of ambient and direct data corresponding to different activities within the multi-tenant environment. Returning to an earlier illustrative example, if ambient and direct data corresponding to a particular room within the multi-tenant environment is not representative of the presence of a user and/or of an activity occurring within the room, the data collection module may generate attribute data corresponding to the lack of activity within the room. This lack of activity, in some instances, may be indicative of a deviation from routine activities and movements within the multi-tenant environment and, thus, may be indicative of an issue or event impacting one or more users within the multi-tenant environment. Thus, through evaluation of the observed data, such as through the aforementioned machine learning algorithm or artificial intelligence, the data collection module, at step 506, may determine whether an event has been detected within the multi-tenant environment.

If the data collection module does not detect an event within the multi-tenant environment, the data collection module may continue to receive and evaluate new observed data from the different devices and/or sensors implemented in the multi-tenant environment, thus restarting the process 500. Alternatively, if the data collection module, through the aforementioned machine learning algorithm or artificial intelligence, determines that the observed data corresponds to an event detected within the multi-tenant environment, the data collection module, at step 508, may aggregate attribute data according to the detected event. For instance, the data collection module may identify any libraries and/or algorithms that may be used to extract attribute data corresponding to different users and events detected within the multi-tenant environment. Using these libraries and/or algorithms, the data collection module may process the observed data to detect physical attributes associated with any user detected within the multi-tenant environment, location attributes corresponding any user detected within the multi-tenant environment, activity attributes corresponding to any events detected within the multi-tenant environment, and timing/pacing attributes corresponding to any detected users and to the events detected within the multi-tenant environment.

As noted above, the data collection module may dynamically train and implement one or more machine learning algorithms or artificial intelligence to extract and aggregate attribute data from the observed data according to the different attribute data categories. Returning to an earlier illustrative example, if the observed data includes audiovisual data, the data collection module may process this audiovisual data using computer vision and/or NLP to obtain data corresponding to the physical attributes of any users detected within the multi-tenant environment and associated with the detected events. As another illustrative example, using computer vision, the data collection module may dynamically evaluate any images and/or video recordings of the multi-tenant environment to calculate a user's location within the multi-tenant environment relative to any of the devices and sensors that collected the observed data within the multi-tenant environment. In yet another illustrative example, the data collection module may implement one or more machine learning algorithms or artificial intelligence to detect, from the observed data, any activities that a user may be engaged in within the multi-tenant environment.

In some instances, the data collection module, as part of step 508, may aggregate the attribute data extracted from the observed data obtained from the various devices and/or sensors according to the corresponding detected event within the multi-tenant environment. For instance, the data collection module may annotate the attribute data according to each attribute category and to the particular detected event identified as being associated with the attribute data. Thus, for a given detected event, the data collection module may include the physical attributes with any detected users, location attributes corresponding to these detected users and the detected event, activity attributes corresponding to the detected event, and the timing/pacing attributes corresponding to the detected users and the occurrence of the detected event. Once the data collection module has aggregated the attribute data for each detected event, the data collection module, at step 510, may transmit the aggregated attribute data to the vector computation system for calculation of an attribute vector usable to identify a specific user associated with the detected event.

It should be noted that the process 500 may be performed continuously and simultaneously for different observed data as the observed data is communicated through the different communications channels or connections between the data collection module and the myriad devices and/or sensors within the multi-tenant environment. For instance, the data collection module may continuously and simultaneously process different data from the different devices and/or sensors in real-time or near real-time as the different data is generated to detect any events occurring within the multi-tenant environment. Further, as these events are detected, the data collection module may continuously and simultaneously extract any attributes associated with these different events and aggregate these attributes for transmission to the vector computation system. Thus, the data collection module may continuously and simultaneously process any amount of data from different sensors and/or devices within the multi-tenant environment in real-time or near real-time as the data is received to dynamically detect events occurring within the multi-tenant environment, identify the attributes associated with these detected events, and aggregate these attributes to generate attribute data that may be converted into attribute vectors for user identification.

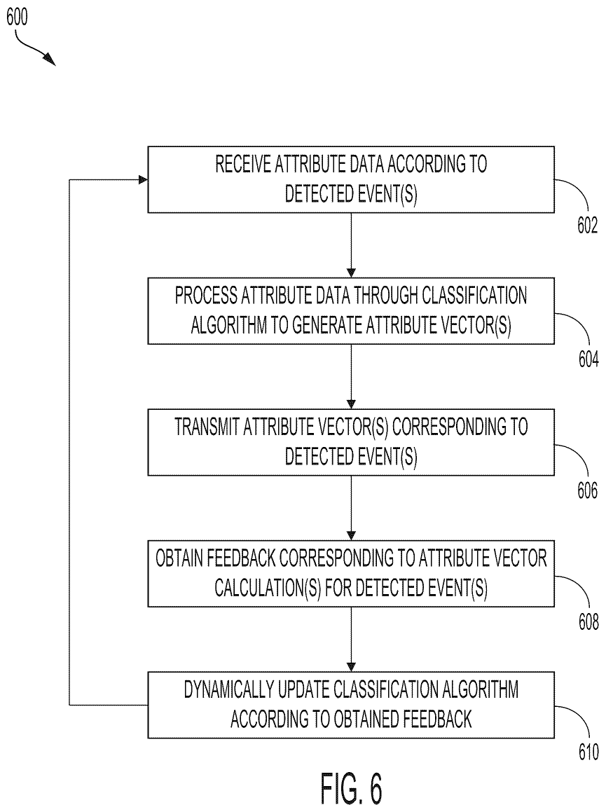

FIG. 6 shows an illustrative example of a process 600 for generating attribute vectors based on received attribute data corresponding to a detected event and for identification of a user associated with the detected event in accordance with at least one embodiment. The process 600 may be performed by the vector computation system (e.g., vector computation system 106 described above in connection with FIGS. 1-4) implemented by the user monitoring gateway. At step 602, the vector computation system receives attribute data corresponding to one or more events detected within the multi-tenant environment. As noted above, when the data collection module implemented by the user monitoring gateway aggregates the attribute data according to a detected event within the multi-tenant environment, the data collection module may automatically transmit this aggregated attribute data to the vector computation system for processing. Accordingly, the vector computation system may receive the aggregated attribute data associated with different events as the aggregated attribute data is generated by the data collection module.

At step 604, the vector computation system may process the aggregated attribution data through a classification algorithm to generate attribute vectors corresponding to the detected events. As noted above, the vector computation system implements and dynamically trains a classification algorithm to convert attribute data from the data collection module into a set of embeddings or vectorial values corresponding to a set of similarity vectors in an n-dimensional space (e.g., n-dimensional vectors). This classification algorithm may be dynamically trained using a dataset of sample attribute data and sample vector values corresponding to the sample attribute data. For example, the classification algorithm may process the sample attribute data included in the dataset to generate an output (e.g., an n-dimensional vector corresponding to each set of attribute data corresponding to a sample event). This output (e.g., the n-dimensional vectors) may be compared to the sample n-dimensional vectors included in the dataset to identify any deviations. Based on these deviations (if any), the classification algorithm may be dynamically, and iteratively, retrained or otherwise updated until an iteration of the classification algorithm is generated that satisfies one or more criteria for generating attribute vectors corresponding to detected events.

Once the vector computation system has converted the received attribute data into a corresponding attribute vector for the particular event, the vector computation system, at step 606, may transmit the attribute vector corresponding to the detected event to a user identification engine implemented by the user monitoring gateway. As described in greater detail herein, the user identification engine may process the attribute vector corresponding to the detected event to determine whether the attribute vector corresponds to a known user associated with the multi-tenant environment.

At step 608, the vector computation system may obtain feedback corresponding to the attribute vector calculations performed by the vector computation system (such as through step 604 described above) for corresponding events. As noted above, feedback obtained by the user identification engine is used to dynamically update one or more machine learning algorithms or artificial intelligence implemented by the user monitoring gateway. This feedback may be used to update the dataset used to dynamically train the classification algorithm by incorporating any updated attribute vectors corresponding to detected events and corresponding users, as well as any annotations indicating how the attributes associated with these events and the users should be converted into n-dimensional attribute vectors. At step 610, the classification algorithm may process this updated dataset to generate a new output that may be evaluated according to the included annotations to determine whether the classification algorithm is accurately converting attribute data into corresponding attribute vectors for event and user identification. Based on this evaluation, the vector computation system may iteratively update the classification algorithm until a new iteration of the classification algorithm is generated that satisfies one or more criteria for generating attribute vectors.

Similar to the process 500 described above, the process 600 may be performed continuously and simultaneously for different attribute data as the attribute data is received from the data collection module and as feedback with regard to attribute vector calculations performed by the vector computation system is received. For instance, the vector computation system may continuously and simultaneously process different attribute data corresponding to different detected events in real-time or near real-time as the different attribute data is generated to generate attribute vectors corresponding to these different events. Further, as feedback is obtained with regard to previously generated attribute vectors corresponding to previously detected events, the vector computation system may continuously and simultaneously retrain or otherwise update the classification algorithm trained to generate attribute vectors based on aggregated attribute data to improve the accuracy of the computations required to generate these attribute vectors. Thus, the vector computation system may continuously and simultaneously process any amount of attribute data corresponding to different events and feedback in real-time or near real-time as the attribute data and feedback are received to dynamically generate attribute vectors corresponding to these different events and, based on obtained feedback, dynamically update the classification algorithm trained to generate these attribute vectors.

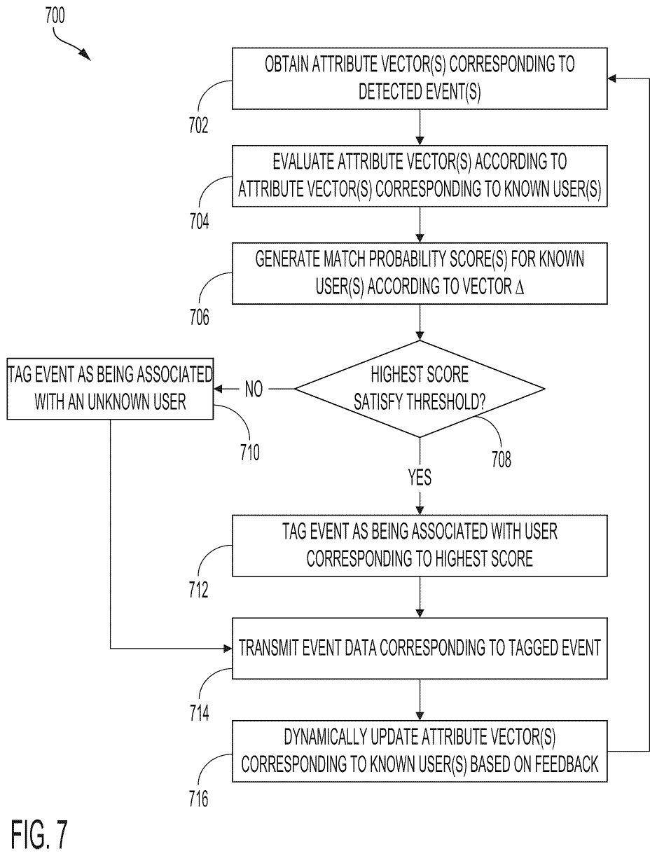

FIG. 7 shows an illustrative example of a process 700 for evaluating an attribute vector corresponding to a detected event against a set of known attribute vectors corresponding to known users within a multi-tenant environment to associate the detected event with a particular user in accordance with at least one embodiment. The process 700 may be performed by the user identification engine (e.g., user identification engine 108 described above in connection with FIGS. 1-4) implemented by the user monitoring gateway. At step 702, the user identification engine obtains one or more attribute vectors corresponding to one or more detected events within a multi-tenant environment. As noted above, when the vector computation system implemented by the user monitoring gateway generates an attribute vector from attribute data according to a detected event within the multi-tenant environment, the vector computation system may automatically transmit this attribute vector to the user identification engine for processing. Accordingly, the user identification engine may receive the attribute vectors associated with different events as these attribute vectors are generated by the vector computation system.

At step 704, the user identification engine may evaluate the one or more attribute vectors according to known user attribute vectors corresponding to users known to be associated with the multi-tenant environment. For instance, the user identification engine may query a known user attributes datastore (such as known user attributes datastore 408 described above in connection with FIG. 4) to retrieve any available attribute vectors corresponding to different users known to be associated with the multi-tenant environment or otherwise associated with the user monitoring gateway implemented in the multi-tenant environment. The attribute vectors associated with these known users may correspond to these users' facial attributes (e.g., facial structure and shape, dental structure, eye color, hair color, facial hair, etc.), vocal attributes (such as through generation of voice signatures, etc.), bodily attributes (e.g., body structure and type, skin color, hair color, etc.), and activities these users may engage in within the multi-tenant environment and when these activities commonly occur. In some instances, the received attribute vectors may be evaluated according to a set of weighting factors corresponding to the sources of attribute data. Returning to an earlier illustrative example, if attribute data corresponding to a user's heart rate is obtained from a wearable device that may be worn by the user and from an embedded pacemaker device that is implanted within the user, the user identification engine may apply greater weight to the attribute data originating from the embedded pacemaker device, as this device may be permanently associated with the user as opposed to the wearable device that may be removed or otherwise used by other users.

At step 706, based on the evaluation of the received attribute vectors according to the known user attribute vectors corresponding to known users within the multi-tenant environment or otherwise associated with the user monitoring gateway, the user identification engine may generate match probability scores for each of these known users according to any vector differences. For instance, the user identification engine may compare each known user attribute vector to a received attribute vector to compute the vectorial value differences (i.e., vector delta or “Δ”) along each dimension of the n-dimensional vector. As noted above, the length of this resultant vector may be inversely proportional to the level of similarity between the received attribute vector and the known user attribute vector corresponding to a particular user. Thus, the user identification engine, through evaluation of the attribute vector against the available known user attribute vectors stored within the known user attributes datastore, may generate different resultant n-dimensional vectors (i.e., vector deltas or “Δ”) for each known user associated with the multi-tenant environment or otherwise associated with the user monitoring gateway. The user identification engine may process these resultant n-dimensional vectors through one or more functions to compute a match probability score for each known user associated with the multi-tenant environment and/or the user monitoring gateway. As noted above, the length of a resultant n-dimensional vector may be inversely proportional to the likelihood of an associated event corresponding to a particular user. For example, a longer resultant n-dimensional vector may serve as an indication that there is little similarity between the attribute vector and the evaluated known user attribute vector associated with a particular user. Alternatively, a shorter or null resultant n-dimensional vector (e.g., a vector having zero length) may denote a greater level of similarity between the attribute vector and the evaluated known user attribute vector associated with a particular user.

At step 708, the user identification engine, based on the resulting match probability scores for the known users associated with the multi-tenant environment and/or the user monitoring gateway, determines whether the highest match probability score satisfies an identification threshold that is used to determine whether the detected event can be associated with a known user. As noted above, this identification threshold may denote a minimum probability score required for a user to be deemed, with a high degree of certainty, to be associated with the detected event. Accordingly, the user identification engine may evaluate each calculated probability score for each known user to identify any match probability scores that satisfy this identification threshold. If the user identification engine determines that no match probability score satisfies the identification threshold (e.g., no match probability score is greater than the required minimum match probability score for identification, etc.), the user identification engine may determine that the detected event cannot be associated with a known user within the multi-tenant environment or otherwise associated with the user monitoring gateway. Accordingly, the user identification engine, at step 710, may tag the detected event to indicate that the detect event corresponds to an unknown user. Alternatively, if the user identification engine determines that the match probability score corresponding to the highest probability of a detected event being associated with a particular user satisfies the identification threshold implemented by the user identification engine, the user identification engine, at step 712, may tag the detected event as being associated with the particular user. Accordingly, the user identification engine may obtain the ambient and direct data associated with the detected event and may annotate this ambient and direct data to indicate that the ambient and direct data is associated with the particular user.

At step 714, the user identification engine may transmit the event data corresponding to the tagged event. For instance, the user identification engine may transmit this event data to one or more entities according to any applicable user preferences or to the configuration of the user monitoring gateway. As noted above, if the user monitoring gateway maintains, for each user in the multi-tenant environment, a set of notification preferences for delivering event data associated with the user, the user identification engine may refer to the set of notification preferences to select the intended one or more recipients of the event data. In some instances, the user identification engine may automatically transmit the event data to a remote user monitoring service, which may process the event data and determine how to route the event data to different intended recipients.

At step 716, the user identification engine may dynamically update the attribute vectors corresponding to different known users based on received feedback. As noted above, the user identification engine may continuously, in real-time or near real-time, receive feedback corresponding to the association of different users (known and unknown users) to detected events within the multi-tenant environment within which the user monitoring gateway is implemented. Returning to an earlier illustrative example, if an administrator of the user monitoring service assigns a particular event to a particular user (such as in response to feedback from the user, a caregiver, a medical professional, etc.), the user identification engine may evaluate the attribute vector corresponding to the particular event and the known attribute vector corresponding to the identified user to determine whether any of the vector values associated with the known attribute vector may be updated to incorporate attributes indicated through the attribute vector associated with the particular event. As another illustrative example, if the detected event is confirmed to be associated with the user identified by the user identification engine, the user identification engine may use the attribute vector calculated for the detected event to supplement the known attribute vector associated with the identified user to improve the likelihood (e.g., user probability scores) of similar attribute vectors associated with different events being linked to the user.

If the user identification engine associated a detected event with an unknown user, and the user identification engine receives feedback indicating an identity of a particular user associated with the detected event, the user identification engine may dynamically update an entry corresponding to this detected event to associate this attribute vector with the identified user. For instance, if the identified user was previously not known to the user monitoring gateway, the user identification engine may automatically update the newly generated entry corresponding to the detected event to associate the entry with the identified user such that new attribute vectors corresponding to newly detected events may be evaluated by the user identification engine against the attribute vector now associated with this identified user to determine whether any of these newly detected events are associated with the identified user as opposed to being classified as being associated with an unknown user.

If the user identification engine previously associated a particular event with an unknown user, and the user identification engine receives feedback indicating that the event is associated with a known user (e.g., a user for which a known user attribute vector is available), the user identification engine may dynamically update this known user attribute vector to incorporate the attribute vector corresponding to the particular event. For instance, the user identification engine may process the known user attribute vector corresponding to the identified user and the attribute vector associated with the particular event through an optimization algorithm to compute the new n-dimensional values for the known user attribute vector, incorporating the detected attributes of the particular event now associated with the identified user.

Feedback corresponding to a particular event and corresponding user identification may include an updated attribute vector corresponding to the particular event. Returning to an earlier illustrative example, if a particular event was previously associated with an unknown or otherwise incorrect user, an administrator of the user monitoring gateway or other evaluating entity (e.g., a user, a caregiver, a medical professional, etc.) may dynamically update the attribute vector associated with the particular event to incorporate one or more attributes associated with the correct user associated with the particular event. The user identification engine may accordingly use this updated attribute vector to supplement an existing attribute vector associated with this user. If the updated attribute vector is associated with a new user not previously known to the user monitoring gateway, the user identification engine may store the updated attribute vector within the known user attributes datastore maintained by the user monitoring gateway.

Similar to the processes 500 and 600 described above, the process 700 may be performed continuously and simultaneously for different attribute vectors as these attribute vectors are received from the vector computation system and as feedback with regard to different events and corresponding user identifications is received. For instance, the user identification engine may continuously and simultaneously process different attribute vectors corresponding to different detected events in real-time or near real-time as the different attribute vectors are generated to identify the users associated with these detected events. Further, as feedback is obtained with regard to previous identification of different users corresponding to previously detected events, the user identification engine may continuously and simultaneously update different attribute vectors corresponding to known users and generate new attribute vectors for any previously unknown users. Thus, the user identification engine may continuously and simultaneously process any attribute vectors corresponding to different events and feedback with regard to the association of different users to detected events in real-time or near real-time as the attribute vectors and feedback are received to dynamically assign different users to newly detected events and, based on the obtained feedback, update existing attribute vectors corresponding to different known users and generate new attribute vectors for users previously unknown to the user monitoring gateway.

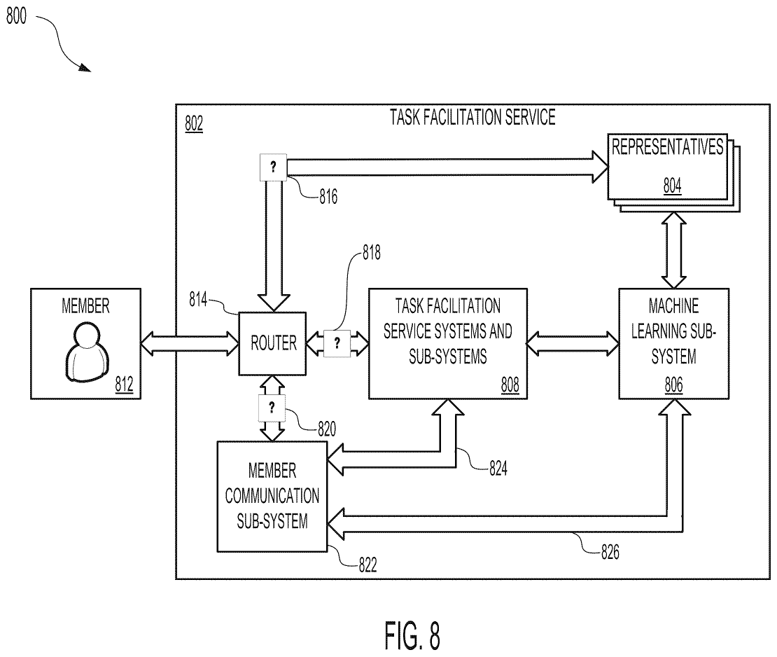

FIG. 8 shows an illustrative example of an environment 800 in which communications with members are processed in accordance with at least one embodiment. In an embodiment, operations performed by representatives 804 are partially and/or fully performed using one or more machine learning algorithms, artificial intelligence systems and/or computational models. For example, as the representatives 804 perform or otherwise coordinate performance of tasks on behalf of a member 812, the task facilitation service 802 may update a profile of the member 812 and/or a computational model of the profile of the member 812.

In an embodiment, as the representatives 804 perform or otherwise coordinate performance of tasks on behalf of a member 812, the task facilitation service 802 updates a profile of the member 812 and/or a computational model of the profile of the member 812 continuously. For example, as a member 812 communicates with a system of the task facilitation service 802, the task facilitation service 802 may update the profile of the member 812 and/or a computational model of the profile of the member 812 continuously during the course of the interaction.

In an embodiment, as the representatives 804 perform or otherwise coordinate performance of tasks on behalf of a member 812, the task facilitation service 802 updates a profile of the member 812 and/or a computational model of the profile of the member 812 dynamically. For example, as a task is performed on behalf of a member 812, a vendor performing the task may provide regular updates to the task facilitation service 802 and the task facilitation service 802 may update the profile of the member 812 and/or a computational model of the profile of the member 812 dynamically at each update from the vendor.

In an embodiment, as the representatives 804 perform or otherwise coordinate performance of tasks on behalf of a member 812, the task facilitation service 802 updates a profile of the member 812 and/or a computational model of the profile of the member 812 automatically. For example, when a proposal is generated for the member, the task facilitation service 802 may update the profile of the member 812 and/or a computational model of the profile of the member 812 automatically as part of the proposal generation process.

In an embodiment, as the representatives 804 perform or otherwise coordinate performance of tasks on behalf of a member 812, the task facilitation service 802 updates a profile of the member 812 and/or a computational model of the profile of the member 812 in real-time. For example, when a member 812 accepts a proposal, the task facilitation service 802 may update the profile of the member 812 and/or a computational model of the profile of the member 812 at the time that the proposal acceptance is provided, rather than delaying the update.

In an embodiment, the task facilitation service 802 updates a profile of the member 812 and/or a computational model of the profile of the member 812 using a machine learning sub-system 806 of the task facilitation service 802. In an embodiment, a machine learning sub-system 806 is a component of the task facilitation service 802 that is configured to implement machine learning algorithms, artificial intelligence systems, and/or computation models. In an example, a machine learning sub-system 806 may use various algorithms to train a machine learning model using sample and/or live data. Additionally, a machine learning sub-system 806 may update the machine learning model as new data is received. In another example, the machine learning sub-system 806 may train and/or update various artificial intelligence systems or generate, train and/or update various computational models. For example, a computational model of the profile of the member 812 may be generated, trained and/or updated by the machine learning sub-system 806 as new information is received about the member 812.

In an embodiment, after the profile of the member 812 and/or a computational model of the profile of the member 812 has been updated over a period of time (e.g., six months, a year, etc.) and/or over a set of tasks (e.g., twenty tasks, thirty tasks, etc.), systems of the task facilitation service 802 (e.g., a task recommendation system) utilize one or more machine learning algorithms, artificial intelligence systems and/or computational models to generate new tasks continuously, automatically, dynamically, and in real-time. For example, the task recommendation system may generate new tasks based on the various attributes of the member's profile (e.g., historical data corresponding to member-representative communications, member feedback corresponding to representative performance and presented tasks/proposals, etc.) with or without representative interaction. In an embodiment, systems of task facilitation service 802 (e.g., a task recommendation system) can automatically communicate with the member 812 to obtain any additional information needed and can also generate proposals that may be presented to the member 812 for performance of these tasks.

In the example illustrated in FIG. 8, communications between the member 812 and the task facilitation service 802 may be routed to one or more entities within the task facilitation service 802. The example illustrated in FIG. 8 shows a communication router 814 (referred to in the illustration as a “router”) however, as may be contemplated and as illustrated in FIG. 8, the router 814 is an abstract representation of one or more techniques for routing communications between entities. Accordingly, communications from the member 812 to the task facilitation service 802 may be routed to one or more entities of the task facilitation service and communications from the one or more entities of the task facilitation service 802 may be routed back to the member 812.

In the example illustrated in FIG. 8, the representatives 804 can monitor communications between task facilitation service systems and/or sub-systems 808 and the member 812 to ensure that the interaction maintains a positive polarity as described herein because the communications can be routed 816 to the representatives 804 and also routed 818 to task facilitation service systems and/or sub-systems 808. For example, if a member 812 is interacting with the task recommendation system, the representatives 804 can determine whether the member 812 is satisfied with the interaction. If the representatives 804 determine that the conversation has a negative polarity (e.g., that the member 812 is not satisfied with the interaction), the representatives 804 may intervene to improve the interaction.

Similarly, other interactions between task facilitation service systems and/or sub-systems 808 and the member 812 may be routed 820 to a member communication sub-system 822 which may be configured to monitor the interactions between task facilitation service systems and/or sub-systems 808 and the member 812. In an embodiment, the member communication sub-system 822 can be configured to intercept the interactions between task facilitation service systems and/or sub-systems 808 and the member 812 (using, for example, the router 814). In such an embodiment, all such interactions can be routed 820 between the member 812 and the member communication sub-system 822 and can be routed 824 between the member communication sub-system 822 and the task facilitation service systems and/or sub-systems 808. In such an embodiment, interactions between the task facilitation service systems and/or sub-systems 808 and the member 812 may not be routed 818 directly. In such an embodiment, the representatives 804 may still monitor interactions between task facilitation service systems and/or sub-systems 808 and the member 812 to ensure that the interaction maintains a positive polarity as described above (e.g., by routing 816 the interactions to the representatives 804).

In an embodiment, the representatives 804 can interact with the machine learning sub-system 806 to update the profile of the member indicating changing member preferences based on an interaction between the representatives 804 the member 812. In an embodiment, the task facilitation service systems and/or sub-systems 808 can interact with the machine learning sub-system 806 to update the profile of the member when, for example, a proposal is accepted or rejected. Additionally, as illustrated in FIG. 8, the interactions between the task facilitation service 802 and the member 812 can be additionally routed 826 between the member communication sub-system 822 and the machine learning sub-system 806. Accordingly, interactions between the member 812 and, for example, a proposal creation sub-system may be used to update the profile of the member as a proposal is created.

Thus, unlike automated customer service systems and environments, wherein the systems and environment may have little or no knowledge of users interacting with agents and/or other automated systems, task facilitation service systems and/or sub-systems 808 can update the profile of the member 812 and/or a computational model of the profile of the member 812 continuously, dynamically, automatically, and/or in real-time. For example, task facilitation service systems and/or sub-systems 808 can update the profile of the member 812 and/or a computational model of the profile of the member 812 using the machine learning sub-system 806 as described herein. Accordingly, task facilitation service systems and/or sub-systems 808 can update the profile of the member 812 and/or a computational model of the profile of the member 812 to provide up-to-date information about the member based on the member's automatic interaction with the task facilitation service 802, based on the member's interaction with the representative 804, and/or based on tasks performed on behalf of the member 812 over time. This information may also be updated continuously, automatically, dynamically, and/or in real-time as tasks and/or proposals are created, proposed, and performed for the member 812. This information may also be used by the task facilitation service 802 to anticipate, identify, and present appropriate or intelligent interactions with the member 812 (e.g., in response to member 812 queries, needs, and/or goals).

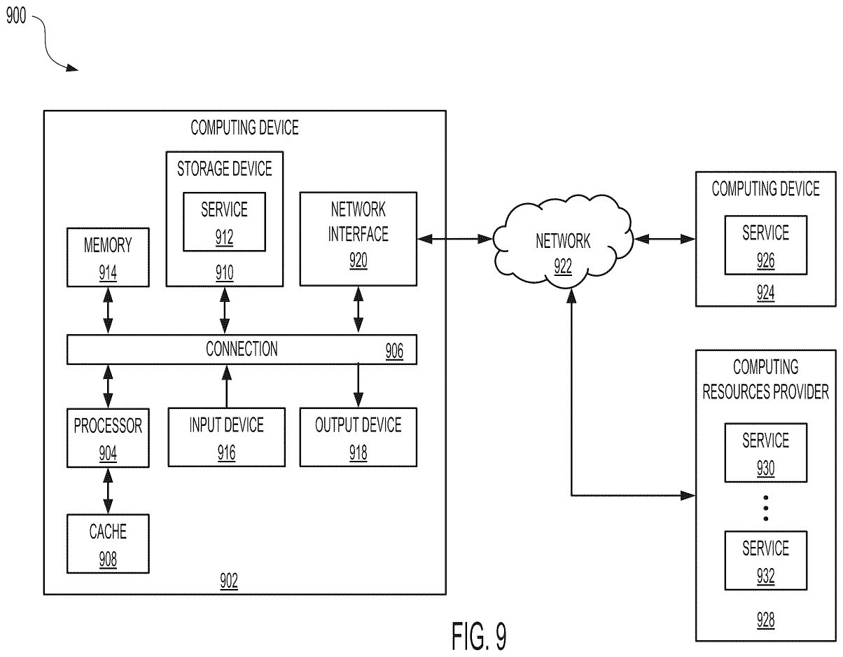

FIG. 9 illustrates a computing system architecture 900, including various components in electrical communication with each other, in accordance with some embodiments. The example computing system architecture 900 illustrated in FIG. 9 includes a computing device 902, which has various components in electrical communication with each other using a connection 906, such as a bus, in accordance with some implementations. The example computing system architecture 900 includes a processing unit 904 that is in electrical communication with various system components, using the connection 906, and including the system memory 914. In some embodiments, the system memory 914 includes read-only memory (ROM), random-access memory (RAM), and other such memory technologies including, but not limited to, those described herein. In some embodiments, the example computing system architecture 900 includes a cache 908 of high-speed memory connected directly with, in close proximity to, or integrated as part of the processor 904. The system architecture 900 can copy data from the memory 914 and/or the storage device 910 to the cache 908 for quick access by the processor 904. In this way, the cache 908 can provide a performance boost that decreases or eliminates processor delays in the processor 904 due to waiting for data. Using modules, methods and services such as those described herein, the processor 904 can be configured to perform various actions. In some embodiments, the cache 908 may include multiple types of cache including, for example, level one (L1) and level two (L2) cache. The memory 914 may be referred to herein as system memory or computer system memory. The memory 914 may include, at various times, elements of an operating system, one or more applications, data associated with the operating system or the one or more applications, or other such data associated with the computing device 902.

Other system memory 914 can be available for use as well. The memory 914 can include multiple different types of memory with different performance characteristics. The processor 904 can include any general purpose processor and one or more hardware or software services, such as service 912 stored in storage device 910, configured to control the processor 904 as well as a special-purpose processor where software instructions are incorporated into the actual processor design. The processor 904 can be a completely self-contained computing system, containing multiple cores or processors, connectors (e.g., buses), memory, memory controllers, caches, etc. In some embodiments, such a self-contained computing system with multiple cores is symmetric. In some embodiments, such a self-contained computing system with multiple cores is asymmetric. In some embodiments, the processor 904 can be a microprocessor, a microcontroller, a digital signal processor (“DSP”), or a combination of these and/or other types of processors. In some embodiments, the processor 904 can include multiple elements such as a core, one or more registers, and one or more processing units such as arithmetic logic units (ALU), floating point units (FPU), graphics processing units (GPU), physics processing units (PPU), neural processing units (NPU), tensor processing units (TPU), digital system processing (DSP) units, or combinations of these and/or other such processing units. The processor 904 can be implemented through different processor architectures, such as Reduced Instruction Set Computing (RISC) architectures (e.g., RISC-V, etc.), Advanced RISC Machine (ARM) architectures, and the like.

The computing device 902 can further include one or more dedicated processors that are configured to exclusively perform machine learning algorithm and/or artificial intelligence calculations and processes. Similar to the processor 904, the one or more dedicated processors implemented on the computing device 902 can be a completely self-contained computing system, containing multiple cores or processors, connectors (e.g., buses), memory, memory controllers, caches, etc. In some embodiments, such a self-contained computing system with multiple cores is symmetric or asymmetric. The one or more dedicated processors can include multiple elements such as a core, one or more registers, and one or more processing units such as ALUs, FPUs, GPUs, PPUs, NPUs, TPUs, DSP units, or combinations of these and/or other such processing units. Similar to the processor 904, these one or more dedicated processors can be implemented through different processor architectures, such as RISC architectures (e.g., RISC-V, etc.), ARM architectures, and the like. This implementation of dedicated processors to exclusively perform machine learning algorithm and/or artificial intelligence calculations and processes provides acceleration of the machine learning algorithm and/or artificial intelligence processes, thereby reducing computing device latency while supporting the operations of the computing device 902 as described herein.

To enable user interaction with the computing system architecture 900, an input device 916 can represent any number of input mechanisms, such as a microphone for speech, a touch-sensitive screen for gesture or graphical input, keyboard, mouse, motion input, pen, and other such input devices. An output device 918 can also be one or more of a number of output mechanisms known to those of skill in the art including, but not limited to, monitors, speakers, printers, haptic devices, and other such output devices. In some instances, multimodal systems can enable a user to provide multiple types of input to communicate with the computing system architecture 900. In some embodiments, the input device 916 and/or the output device 918 can be coupled to the computing device 902 using a remote connection device such as, for example, a communication interface such as the network interface 920 described herein. In such embodiments, the communication interface can govern and manage the input and output received from the attached input device 916 and/or output device 918. As may be contemplated, there is no restriction on operating on any particular hardware arrangement and accordingly the basic features here may easily be substituted for other hardware, software, or firmware arrangements as they are developed.

In some embodiments, the storage device 910 can be described as non-volatile storage or non-volatile memory. Such non-volatile memory or non-volatile storage can be a hard disk or other types of computer readable media which can store data that are accessible by a computer, such as magnetic cassettes, flash memory cards, solid state memory devices, digital versatile disks, cartridges, RAM, ROM, and hybrids thereof.

As described above, the storage device 910 can include hardware and/or software services such as service 912 that can control or configure the processor 904 to perform one or more functions including, but not limited to, the methods, processes, functions, systems, and services described herein in various embodiments. In some embodiments, the hardware or software services can be implemented as modules. As illustrated in example computing system architecture 900, the storage device 910 can be connected to other parts of the computing device 902 using the system connection 906. In an embodiment, a hardware service or hardware module such as service 912, that performs a function can include a software component stored in a non-transitory computer-readable medium that, in connection with the necessary hardware components, such as the processor 904, connection 906, cache 908, storage device 910, memory 914, input device 916, output device 918, and so forth, can carry out the functions such as those described herein.

The disclosed processed for generating and executing experience recommendations can be performed using a computing system such as the example computing system illustrated in FIG. 9, using one or more components of the example computing system architecture 900. An example computing system can include a processor (e.g., a central processing unit), memory, non-volatile memory, and an interface device. The memory may store data and/or and one or more code sets, software, scripts, etc. The components of the computer system can be coupled together via a bus or through some other known or convenient device.

In some embodiments, the processor can be configured to carry out some or all of methods and functions for generating and executing experience recommendations described herein by, for example, executing code using a processor such as processor 904 wherein the code is stored in memory such as memory 914 as described herein. One or more of a user device, a provider server or system, a database system, or other such devices, services, or systems may include some or all of the components of the computing system such as the example computing system illustrated in FIG. 9, using one or more components of the example computing system architecture 900 illustrated herein. As may be contemplated, variations on such systems can be considered as within the scope of the present disclosure.

This disclosure contemplates the computer system taking any suitable physical form. As example and not by way of limitation, the computer system can be an embedded computer system, a system-on-chip (SOC), a single-board computer system (SBC) (such as, for example, a computer-on-module (COM) or system-on-module (SOM)), a desktop computer system, a laptop or notebook computer system, a tablet computer system, a wearable computer system or interface, an interactive kiosk, a mainframe, a mesh of computer systems, a mobile telephone, a personal digital assistant (PDA), a server, or a combination of two or more of these. Where appropriate, the computer system may include one or more computer systems; be unitary or distributed; span multiple locations; span multiple machines; and/or reside in a cloud computing system which may include one or more cloud components in one or more networks as described herein in association with the computing resources provider 928. Where appropriate, one or more computer systems may perform without substantial spatial or temporal limitation one or more steps of one or more methods described or illustrated herein. As an example and not by way of limitation, one or more computer systems may perform in real time or in batch mode one or more steps of one or more methods described or illustrated herein. One or more computer systems may perform at different times or at different locations one or more steps of one or more methods described or illustrated herein, where appropriate.

The processor 904 can be a conventional microprocessor such as an Intel® microprocessor, an AMD® microprocessor, a Motorola® microprocessor, or other such microprocessors. The processor 1104 can include multiple elements such as a core, one or more registers, and one or more processing units such as ALUs, FPUs, GPUs, PPUs, NPUs, TPUs, DSP units, or combinations of these and/or other such processing units. The processor 904, as noted above, can be implemented through different processor architectures, such as RISC architectures (e.g., RISC-V, etc.), ARM architectures, and the like. One of skill in the relevant art will recognize that the terms “machine-readable (storage) medium” or “computer-readable (storage) medium” include any type of device that is accessible by the processor.

The memory 914 can be coupled to the processor 904 by, for example, a connector such as connector 906, or a bus. As used herein, a connector or bus such as connector 906 is a communications system that transfers data between components within the computing device 902 and may, in some embodiments, be used to transfer data between computing devices. The connector 906 can be a data bus, a memory bus, a system bus, or other such data transfer mechanism. Examples of such connectors include, but are not limited to, an industry standard architecture (ISA” bus, an extended ISA (EISA) bus, a parallel AT attachment (PATA” bus (e.g., an integrated drive electronics (IDE) or an extended IDE (EIDE) bus), or the various types of parallel component interconnect (PCI) buses (e.g., PCI, PCIe, PCI-104, etc.).

The memory 914 can include RAM including, but not limited to, dynamic RAM (DRAM), static RAM (SRAM), synchronous dynamic RAM (SDRAM), non-volatile random access memory (NVRAM), and other types of RAM. The DRAM may include error-correcting code (EEC). The memory can also include ROM including, but not limited to, programmable ROM (PROM), erasable and programmable ROM (EPROM), electronically erasable and programmable ROM (EEPROM), Flash Memory, masked ROM (MROM), and other types or ROM. The memory 914 can also include magnetic or optical data storage media including read-only (e.g., CD ROM and DVD ROM) or otherwise (e.g., CD or DVD). The memory can be local, remote, or distributed.

As described above, the connector 906 (or bus) can also couple the processor 904 to the storage device 910, which may include non-volatile memory or storage and which may also include a drive unit. In some embodiments, the non-volatile memory or storage is a magnetic floppy or hard disk, a magnetic-optical disk, an optical disk, a ROM (e.g., a CD-ROM, DVD-ROM, EPROM, or EEPROM), a magnetic or optical card, or another form of storage for data. Some of this data is may be written, by a direct memory access process, into memory during execution of software in a computer system. The non-volatile memory or storage can be local, remote, or distributed. In some embodiments, the non-volatile memory or storage is optional. As may be contemplated, a computing system can be created with all applicable data available in memory. A typical computer system will usually include at least one processor, memory, and a device (e.g., a bus) coupling the memory to the processor.

Software and/or data associated with software can be stored in the non-volatile memory and/or the drive unit. In some embodiments (e.g., for large programs) it may not be possible to store the entire program and/or data in the memory at any one time. In such embodiments, the program and/or data can be moved in and out of memory from, for example, an additional storage device such as storage device 910. Nevertheless, it should be understood that for software to run, if necessary, it is moved to a computer readable location appropriate for processing, and for illustrative purposes, that location is referred to as the memory herein. Even when software is moved to the memory for execution, the processor can make use of hardware registers to store values associated with the software, and local cache that, ideally, serves to speed up execution. As used herein, a software program is assumed to be stored at any known or convenient location (from non-volatile storage to hardware registers), when the software program is referred to as “implemented in a computer-readable medium.” A processor is considered to be “configured to execute a program” when at least one value associated with the program is stored in a register readable by the processor.

The connection 906 can also couple the processor 904 to a network interface device such as the network interface 920. The interface can include one or more of a modem or other such network interfaces including, but not limited to those described herein. It will be appreciated that the network interface 920 may be considered to be part of the computing device 902 or may be separate from the computing device 902. The network interface 920 can include one or more of an analog modem, Integrated Services Digital Network (ISDN) modem, cable modem, token ring interface, satellite transmission interface, or other interfaces for coupling a computer system to other computer systems. In some embodiments, the network interface 920 can include one or more input and/or output (I/O) devices. The I/O devices can include, by way of example but not limitation, input devices such as input device 916 and/or output devices such as output device 918. For example, the network interface 920 may include a keyboard, a mouse, a printer, a scanner, a display device, and other such components. Other examples of input devices and output devices are described herein. In some embodiments, a communication interface device can be implemented as a complete and separate computing device.

In operation, the computer system can be controlled by operating system software that includes a file management system, such as a disk operating system. One example of operating system software with associated file management system software is the family of Windows® operating systems and their associated file management systems. Another example of operating system software with its associated file management system software is the Linux™ operating system and its associated file management system including, but not limited to, the various types and implementations of the Linux® operating system and their associated file management systems. The file management system can be stored in the non-volatile memory and/or drive unit and can cause the processor to execute the various acts required by the operating system to input and output data and to store data in the memory, including storing files on the non-volatile memory and/or drive unit. As may be contemplated, other types of operating systems such as, for example, MacOS®, other types of UNIX® operating systems (e.g., BSD™ and decendents, Xenix™, SunOS™, HP-UX®, etc.), mobile operating systems (e.g., iOS® and variants, Chrome®, Ubuntu Touch®, watchOS®, Windows 10 Mobile®, the Blackberry® OS, etc.), and real-time operating systems (e.g., VxWorks®, QNX®, eCos®, RTLinux®, etc.) may be considered as within the scope of the present disclosure. As may be contemplated, the names of operating systems, mobile operating systems, real-time operating systems, languages, and devices, listed herein may be registered trademarks, service marks, or designs of various associated entities.

In some embodiments, the computing device 902 can be connected to one or more additional computing devices such as computing device 924 via a network 922 using a connection such as the network interface 920. In such embodiments, the computing device 924 may execute one or more services 926 to perform one or more functions under the control of, or on behalf of, programs and/or services operating on computing device 902. In some embodiments, a computing device such as computing device 924 may include one or more of the types of components as described in connection with computing device 902 including, but not limited to, a processor such as processor 904, dedicated processors to exclusively perform and accelerate machine learning algorithm and/or artificial intelligence calculations and processes, a connection such as connection 906, a cache such as cache 908, a storage device such as storage device 910, memory such as memory 914, an input device such as input device 916, and an output device such as output device 918. In such embodiments, the computing device 924 can carry out the functions such as those described herein in connection with computing device 902. In some embodiments, the computing device 902 can be connected to a plurality of computing devices such as computing device 924, each of which may also be connected to a plurality of computing devices such as computing device 924. Such an embodiment may be referred to herein as a distributed computing environment.

The network 922 can be any network including an internet, an intranet, an extranet, a cellular network, a Wi-Fi network, a local area network (LAN), a wide area network (WAN), a satellite network, a Bluetooth® or Bluetooth® Low Energy (BLE) network, a virtual private network (VPN), a public switched telephone network, an infrared (IR) network, an internet of things (IoT network), a Zigbee network, a Z-Wave network, or any other such network or combination of networks. Communications via the network 922 can be wired connections, wireless connections, or combinations thereof. Communications via the network 922 can be made via a variety of communications protocols including, but not limited to, Transmission Control Protocol/Internet Protocol (TCP/IP), User Datagram Protocol (UDP), protocols in various layers of the Open System Interconnection (OSI) model, File Transfer Protocol (FTP), Universal Plug and Play (UPnP), Network File System (NFS), Server Message Block (SMB), Common Internet File System (CIFS), Z-Wave, Zigbee, Matter, Message Queuing Telemetry Transport (MQTT), WebSocket, and other such communications protocols.

Communications over the network 922, within the computing device 902, within the computing device 924, or within the computing resources provider 928 can include information, which also may be referred to herein as content. The information may include text, graphics, audio, video, haptics, and/or any other information that can be provided to a user of the computing device such as the computing device 902. In an embodiment, the information can be delivered using a transfer protocol such as Hypertext Markup Language (HTML), Extensible Markup Language (XML), JavaScript®, Cascading Style Sheets (CSS), JavaScript® Object Notation (JSON), Protocol Buffers (Protobuf), MQTT, Matter, and other such protocols and/or structured languages, including any variants implementing Secure Sockets Layer (SSL), Transport Layer Security (TLS), or any other security protocols. and other such protocols and/or structured languages. The information may first be processed by the computing device 902 and presented to a user of the computing device 902 using forms that are perceptible via sight, sound, smell, taste, touch, or other such mechanisms. In some embodiments, communications over the network 922 can be received and/or processed by a computing device configured as a server. Such communications can be sent and received using PHP: Hypertext Preprocessor (“PHP”), Python™, Ruby, Perl® and variants, Java®, HTML, XML, Swift, Kotlin, WebASM, or another such server-side processing language.

In some embodiments, the computing device 902 and/or the computing device 924 can be connected to a computing resources provider 928 via the network 922 using a network interface such as those described herein (e.g. network interface 920). In such embodiments, one or more systems (e.g., service 930 and service 932) hosted within the computing resources provider 928 (also referred to herein as within “a computing resources provider environment”) may execute one or more services to perform one or more functions under the control of, or on behalf of, programs and/or services operating on computing device 902 and/or computing device 924. Systems such as service 930 and service 932 may include one or more computing devices such as those described herein to execute computer code to perform the one or more functions under the control of, or on behalf of, programs and/or services operating on computing device 902 and/or computing device 924.

For example, the computing resources provider 928 may provide a service, operating on service 930 to store data for the computing device 902 when, for example, the amount of data that the computing device 902 exceeds the capacity of storage device 910. In another example, the computing resources provider 928 may provide a service to first instantiate a virtual machine (VM) on service 932, use that VM to access the data stored on service 932, perform one or more operations on that data, and provide a result of those one or more operations to the computing device 902. Such operations (e.g., data storage and VM instantiation) may be referred to herein as operating “in the cloud,” “within a cloud computing environment,” or “within a hosted virtual machine environment,” and the computing resources provider 928 may also be referred to herein as “the cloud.” Examples of such computing resources providers include, but are not limited to Amazon® Web Services (AWS®), Microsoft's Azure®, IBM Cloud®, Google Cloud®, Oracle Cloud® etc. Computing resources provider 928 may also be provided on-premises or in a hybrid cloud computing environment.

Services provided by a computing resources provider 928 include, but are not limited to, data analytics, data storage, archival storage, big data storage, virtual computing (including various scalable VM architectures), blockchain services, containers (e.g., application encapsulation), database services, development environments (including sandbox development environments), e-commerce solutions, game services, media and content management services, security services, serverless hosting, virtual reality (VR) systems, and augmented reality (AR) systems. Various techniques to facilitate such services include, but are not limited to, virtual machines, virtual storage, database services, system schedulers (e.g., hypervisors), resource management systems, various types of short-term, mid-term, long-term, and archival storage devices, etc.

As may be contemplated, the systems such as service 930 and service 932 may implement versions of various services (e.g., the service 912 or the service 926) on behalf of, or under the control of, computing device 902 and/or computing device 924. Such implemented versions of various services may involve one or more virtualization techniques so that, for example, it may appear to a user of computing device 902 that the service 912 is executing on the computing device 902 when the service is executing on, for example, service 930. As may also be contemplated, the various services operating within the computing resources provider 928 environment may be distributed among various systems within the environment as well as partially distributed onto computing device 924 and/or computing device 902.

Client devices, user devices, computer resources provider devices, network devices, and other devices can be computing systems that include one or more integrated circuits, input devices, output devices, data storage devices, and/or network interfaces, among other things. The integrated circuits can include, for example, one or more processors, volatile memory, and/or non-volatile memory, among other things such as those described herein. The input devices can include, for example, a keyboard, a mouse, a key pad, a touch interface, a microphone, a camera, and/or other types of input devices including, but not limited to, those described herein. The output devices can include, for example, a display screen, a speaker, a haptic feedback system, a printer, and/or other types of output devices including, but not limited to, those described herein. A data storage device, such as a hard drive or flash memory, can enable the computing device to temporarily or permanently store data. A network interface, such as a wireless or wired interface, can enable the computing device to communicate with a network. Examples of computing devices (e.g., the computing device 902) include, but is not limited to, desktop computers, laptop computers, server computers, hand-held computers, tablets, smart phones, personal digital assistants, digital home assistants, wearable devices, smart devices, and combinations of these and/or other such computing devices as well as machines and apparatuses in which a computing device has been incorporated and/or virtually implemented.

The techniques described herein may also be implemented in electronic hardware, computer software, firmware, or any combination thereof. Such techniques may be implemented in any of a variety of devices such as general purposes computers, wireless communication device handsets, or integrated circuit devices having multiple uses including application in wireless communication device handsets and other devices. Any features described as modules or components may be implemented together in an integrated logic device or separately as discrete but interoperable logic devices. If implemented in software, the techniques may be realized at least in part by a computer-readable data storage medium comprising program code including instructions that, when executed, performs one or more of the methods described above. The computer-readable data storage medium may form part of a computer program product, which may include packaging materials. The computer-readable medium may comprise memory or data storage media, such as that described herein. The techniques additionally, or alternatively, may be realized at least in part by a computer-readable communication medium that carries or communicates program code in the form of instructions or data structures and that can be accessed, read, and/or executed by a computer, such as propagated signals or waves.

The program code may be executed by a processor, which may include one or more processors, such as one or more digital signal processors (DSPs), general purpose microprocessors, an application specific integrated circuits (ASICs), field programmable logic arrays (FPGAs), or other equivalent integrated or discrete logic circuitry. Such a processor may be configured to perform any of the techniques described in this disclosure. A general purpose processor may be a microprocessor; but in the alternative, the processor may be any conventional processor, controller, microcontroller, or state machine. A processor may also be implemented as a combination of computing devices (e.g., a combination of a DSP and a microprocessor), a plurality of microprocessors, one or more microprocessors in conjunction with a DSP core, or any other such configuration. Accordingly, the term “processor,” as used herein may refer to any of the foregoing structure, any combination of the foregoing structure, or any other structure or apparatus suitable for implementation of the techniques described herein. In addition, in some aspects, the functionality described herein may be provided within dedicated software modules or hardware modules configured for implementing a suspended database update system.

As used herein, the term “machine-readable media” and equivalent terms “machine-readable storage media,” “computer-readable media,” and “computer-readable storage media” refer to media that includes, but is not limited to, portable or non-portable storage devices, optical storage devices, removable or non-removable storage devices, and various other mediums capable of storing, containing, or carrying instruction(s) and/or data. A computer-readable medium may include a non-transitory medium in which data can be stored and that does not include carrier waves and/or transitory electronic signals propagating wirelessly or over wired connections. Examples of a non-transitory medium may include, but are not limited to, a magnetic disk or tape, optical storage media such as compact disk (CD) or digital versatile disk (DVD), solid state drives (SSD), flash memory, memory or memory devices.

A machine-readable medium or machine-readable storage medium may have stored thereon code and/or machine-executable instructions that may represent a procedure, a function, a subprogram, a program, a routine, a subroutine, a module, a software package, a class, or any combination of instructions, data structures, or program statements. A code segment may be coupled to another code segment or a hardware circuit by passing and/or receiving information, data, arguments, parameters, or memory contents. Information, arguments, parameters, data, etc. may be passed, forwarded, or transmitted via any suitable means including memory sharing, message passing, token passing, network transmission, or the like. Further examples of machine-readable storage media, machine-readable media, or computer-readable (storage) media include but are not limited to recordable type media such as volatile and non-volatile memory devices, floppy and other removable disks, hard disk drives, optical disks (e.g., CDs, DVDs, etc.), among others, and transmission type media such as digital and analog communication links.

As may be contemplated, while examples herein may illustrate or refer to a machine-readable medium or machine-readable storage medium as a single medium, the term “machine-readable medium” and “machine-readable storage medium” should be taken to include a single medium or multiple media (e.g., a centralized or distributed database, and/or associated caches and servers) that store the one or more sets of instructions. The term “machine-readable medium” and “machine-readable storage medium” shall also be taken to include any medium that is capable of storing, encoding, or carrying a set of instructions for execution by the system and that cause the system to perform any one or more of the methodologies or modules of disclosed herein.

Some portions of the detailed description herein may be presented in terms of algorithms and symbolic representations of operations on data bits within a computer memory. These algorithmic descriptions and representations are the means used by those skilled in the data processing arts to most effectively convey the substance of their work to others skilled in the art. An algorithm is here, and generally, conceived to be a self-consistent sequence of operations leading to a desired result. The operations are those requiring physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise as apparent from the following discussion, it is appreciated that throughout the description, discussions utilizing terms such as “processing” or “computing” or “calculating” or “determining” or “displaying” or “generating” or the like, refer to the action and processes of a computer system, or similar electronic computing device, that manipulates and transforms data represented as physical (electronic) quantities within registers and memories of the computer system into other data similarly represented as physical quantities within the computer system memories or registers or other such information storage, transmission or display devices.

It is also noted that individual implementations may be described as a process which is depicted as a flowchart, a flow diagram, a data flow diagram, a structure diagram, or a block diagram (e.g., the processes illustrated in FIGS. 6-7). Although a flowchart, a flow diagram, a data flow diagram, a structure diagram, or a block diagram may describe the operations as a sequential process, many of the operations can be performed in parallel or concurrently. In addition, the order of the operations may be re-arranged. A process illustrated in a figure is terminated when its operations are completed, but could have additional steps not included in the figure. A process may correspond to a method, a function, a procedure, a subroutine, a subprogram, etc. When a process corresponds to a function, its termination can correspond to a return of the function to the calling function or the main function.

In some embodiments, one or more implementations of an algorithm such as those described herein may be implemented using a machine learning or artificial intelligence algorithm. Such a machine learning or artificial intelligence algorithm may be trained using supervised, unsupervised, reinforcement, or other such training techniques. For example, a set of data may be analyzed using one of a variety of machine learning algorithms to identify correlations between different elements of the set of data without supervision and feedback (e.g., an unsupervised training technique). A machine learning data analysis algorithm may also be trained using sample or live data to identify potential correlations. Such algorithms may include k-means clustering algorithms, fuzzy c-means (FCM) algorithms, expectation-maximization (EM) algorithms, hierarchical clustering algorithms, density-based spatial clustering of applications with noise (DBSCAN) algorithms, and the like. Other examples of machine learning or artificial intelligence algorithms include, but are not limited to, genetic algorithms, backpropagation, reinforcement learning, decision trees, liner classification, artificial neural networks, anomaly detection, and such. More generally, machine learning or artificial intelligence methods may include regression analysis, dimensionality reduction, metalearning, reinforcement learning, deep learning, and other such algorithms and/or methods. As may be contemplated, the terms “machine learning” and “artificial intelligence” are frequently used interchangeably due to the degree of overlap between these fields and many of the disclosed techniques and algorithms have similar approaches.

As an example of a supervised training technique, a set of data can be selected for training of the machine learning model to facilitate identification of correlations between members of the set of data. The machine learning model may be evaluated to determine, based on the sample inputs supplied to the machine learning model, whether the machine learning model is producing accurate correlations between members of the set of data. Based on this evaluation, the machine learning model may be modified to increase the likelihood of the machine learning model identifying the desired correlations. The machine learning model may further be dynamically trained by soliciting feedback from users of a system as to the efficacy of correlations provided by the machine learning algorithm or artificial intelligence model (i.e., the supervision). The machine learning algorithm or artificial intelligence model may use this feedback to improve the algorithm for generating correlations (e.g., the feedback may be used to further train the machine learning algorithm or artificial intelligence model to provide more accurate correlations).

The various examples of flowcharts, flow diagrams, data flow diagrams, structure diagrams, or block diagrams discussed herein may further be implemented by hardware, software, firmware, middleware, microcode, hardware description languages, or any combination thereof. When implemented in software, firmware, middleware or microcode, the program code or code segments to perform the necessary tasks (e.g., a computer-program product) may be stored in a computer-readable or machine-readable storage medium (e.g., a medium for storing program code or code segments) such as those described herein. A processor(s), implemented in an integrated circuit, may perform the necessary tasks.

The various illustrative logical blocks, modules, circuits, and algorithm steps described in connection with the implementations disclosed herein may be implemented as electronic hardware, computer software, firmware, or combinations thereof. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present disclosure.

It should be noted, however, that the algorithms and displays presented herein are not inherently related to any particular computer or other apparatus. Various general purpose systems may be used with programs in accordance with the teachings herein, or it may prove convenient to construct more specialized apparatus to perform the methods of some examples. The required structure for a variety of these systems will appear from the description below. In addition, the techniques are not described with reference to any particular programming language, and various examples may thus be implemented using a variety of programming languages.

In various implementations, the system operates as a standalone device or may be connected (e.g., networked) to other systems. In a networked deployment, the system may operate in the capacity of a server or a client system in a client-server network environment, or as a peer system in a peer-to-peer (or distributed) network environment.

The system may be a server computer, a client computer, a personal computer (PC), a tablet PC (e.g., an iPad®, a Microsoft Surface®, a Chromebook®, etc.), a laptop computer, a set-top box (STB), a personal digital assistant (PDA), a mobile device (e.g., a cellular telephone, an iPhone®, and Android® device, a Blackberry®, etc.), a wearable device, an embedded computer system, an electronic book reader, a processor, a telephone, a web appliance, a network router, switch or bridge, or any system capable of executing a set of instructions (sequential or otherwise) that specify actions to be taken by that system. The system may also be a virtual system such as a virtual version of one of the aforementioned devices that may be hosted on another computer device such as the computer device 902.

In general, the routines executed to implement the implementations of the disclosure, may be implemented as part of an operating system or a specific application, component, program, object, module or sequence of instructions referred to as “computer programs.” The computer programs typically comprise one or more instructions set at various times in various memory and storage devices in a computer, and that, when read and executed by one or more processing units or processors in a computer, cause the computer to perform operations to execute elements involving the various aspects of the disclosure.

Moreover, while examples have been described in the context of fully functioning computers and computer systems, those skilled in the art will appreciate that the various examples are capable of being distributed as a program object in a variety of forms, and that the disclosure applies equally regardless of the particular type of machine or computer-readable media used to actually effect the distribution.

In some circumstances, operation of a memory device, such as a change in state from a binary one to a binary zero or vice-versa, for example, may comprise a transformation, such as a physical transformation. With particular types of memory devices, such a physical transformation may comprise a physical transformation of an article to a different state or thing. For example, but without limitation, for some types of memory devices, a change in state may involve an accumulation and storage of charge or a release of stored charge. Likewise, in other memory devices, a change of state may comprise a physical change or transformation in magnetic orientation or a physical change or transformation in molecular structure, such as from crystalline to amorphous or vice versa. The foregoing is not intended to be an exhaustive list of all examples in which a change in state for a binary one to a binary zero or vice-versa in a memory device may comprise a transformation, such as a physical transformation. Rather, the foregoing is intended as illustrative examples.

A storage medium typically may be non-transitory or comprise a non-transitory device. In this context, a non-transitory storage medium may include a device that is tangible, meaning that the device has a concrete physical form, although the device may change its physical state. Thus, for example, non-transitory refers to a device remaining tangible despite this change in state.

The above description and drawings are illustrative and are not to be construed as limiting or restricting the subject matter to the precise forms disclosed. Persons skilled in the relevant art can appreciate that many modifications and variations are possible in light of the above disclosure and may be made thereto without departing from the broader scope of the embodiments as set forth herein. Numerous specific details are described to provide a thorough understanding of the disclosure. However, in certain instances, well-known or conventional details are not described in order to avoid obscuring the description.

As used herein, the terms “connected,” “coupled,” or any variant thereof when applying to modules of a system, means any connection or coupling, either direct or indirect, between two or more elements; the coupling of connection between the elements can be physical, logical, or any combination thereof. Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number respectively. The word “or,” in reference to a list of two or more items, covers all of the following interpretations of the word: any of the items in the list, all of the items in the list, or any combination of the items in the list.

As used herein, the terms “a” and “an” and “the” and other such singular referents are to be construed to include both the singular and the plural, unless otherwise indicated herein or clearly contradicted by context.

As used herein, the terms “comprising,” “having,” “including,” and “containing” are to be construed as open-ended (e.g., “including” is to be construed as “including, but not limited to”), unless otherwise indicated or clearly contradicted by context.

As used herein, the recitation of ranges of values is intended to serve as a shorthand method of referring individually to each separate value falling within the range, unless otherwise indicated or clearly contradicted by context. Accordingly, each separate value of the range is incorporated into the specification as if it were individually recited herein.

As used herein, use of the terms “set” (e.g., “a set of items”) and “subset” (e.g., “a subset of the set of items”) is to be construed as a nonempty collection including one or more members unless otherwise indicated or clearly contradicted by context. Furthermore, unless otherwise indicated or clearly contradicted by context, the term “subset” of a corresponding set does not necessarily denote a proper subset of the corresponding set but that the subset and the set may include the same elements (i.e., the set and the subset may be the same).

As used herein, use of conjunctive language such as “at least one of A, B, and C” is to be construed as indicating one or more of A, B, and C (e.g., any one of the following nonempty subsets of the set {A, B, C}, namely: {A}, {B}, {C}, {A, B}, {A, C}, {B, C}, or {A, B, C}) unless otherwise indicated or clearly contradicted by context. Accordingly, conjunctive language such as “as least one of A, B, and C” does not imply a requirement for at least one of A, at least one of B, and at least one of C.

As used herein, the use of examples or exemplary language (e.g., “such as” or “as an example”) is intended to more clearly illustrate embodiments and does not impose a limitation on the scope unless otherwise claimed. Such language in the specification should not be construed as indicating any non-claimed element is required for the practice of the embodiments described and claimed in the present disclosure.

As used herein, where components are described as being “configured to” perform certain operations, such configuration can be accomplished, for example, by designing electronic circuits or other hardware to perform the operation, by programming programmable electronic circuits (e.g., microprocessors, or other suitable electronic circuits) to perform the operation, or any combination thereof.

Those of skill in the art will appreciate that the disclosed subject matter may be embodied in other forms and manners not shown below. It is understood that the use of relational terms, if any, such as first, second, top and bottom, and the like are used solely for distinguishing one entity or action from another, without necessarily requiring or implying any such actual relationship or order between such entities or actions.

While processes or blocks are presented in a given order, alternative implementations may perform routines having steps, or employ systems having blocks, in a different order, and some processes or blocks may be deleted, moved, added, subdivided, substituted, combined, and/or modified to provide alternative or sub combinations. Each of these processes or blocks may be implemented in a variety of different ways. Also, while processes or blocks are at times shown as being performed in series, these processes or blocks may instead be performed in parallel, or may be performed at different times. Further any specific numbers noted herein are only examples: alternative implementations may employ differing values or ranges.

The teachings of the disclosure provided herein can be applied to other systems, not necessarily the system described above. The elements and acts of the various examples described above can be combined to provide further examples.

Any patents and applications and other references noted above, including any that may be listed in accompanying filing papers, are incorporated herein by reference. Aspects of the disclosure can be modified, if necessary, to employ the systems, functions, and concepts of the various references described above to provide yet further examples of the disclosure.

These and other changes can be made to the disclosure in light of the above Detailed Description. While the above description describes certain examples, and describes the best mode contemplated, no matter how detailed the above appears in text, the teachings can be practiced in many ways. Details of the system may vary considerably in its implementation details, while still being encompassed by the subject matter disclosed herein. As noted above, particular terminology used when describing certain features or aspects of the disclosure should not be taken to imply that the terminology is being redefined herein to be restricted to any specific characteristics, features, or aspects of the disclosure with which that terminology is associated. In general, the terms used in the following claims should not be construed to limit the disclosure to the specific implementations disclosed in the specification, unless the above Detailed Description section explicitly defines such terms. Accordingly, the actual scope of the disclosure encompasses not only the disclosed implementations, but also all equivalent ways of practicing or implementing the disclosure under the claims.

While certain aspects of the disclosure are presented below in certain claim forms, the inventors contemplate the various aspects of the disclosure in any number of claim forms. Any claims intended to be treated under 35 U.S.C. § 112(f) will begin with the words “means for”. Accordingly, the applicant reserves the right to add additional claims after filing the application to pursue such additional claim forms for other aspects of the disclosure.

The terms used in this specification generally have their ordinary meanings in the art, within the context of the disclosure, and in the specific context where each term is used. Certain terms that are used to describe the disclosure are discussed above, or elsewhere in the specification, to provide additional guidance to the practitioner regarding the description of the disclosure. For convenience, certain terms may be highlighted, for example using capitalization, italics, and/or quotation marks. The use of highlighting has no influence on the scope and meaning of a term; the scope and meaning of a term is the same, in the same context, whether or not it is highlighted. It will be appreciated that same element can be described in more than one way.

Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and is not intended to further limit the scope and meaning of the disclosure or of any exemplified term. Likewise, the disclosure is not limited to various examples given in this specification.

Without intent to further limit the scope of the disclosure, examples of instruments, apparatus, methods and their related results according to the examples of the present disclosure are given below. Note that titles or subtitles may be used in the examples for convenience of a reader, which in no way should limit the scope of the disclosure. Unless otherwise defined, all technical and scientific terms used herein have the same meaning as commonly understood by one of ordinary skill in the art to which this disclosure pertains. In the case of conflict, the present document, including definitions will control.

Some portions of this description describe examples in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like. Furthermore, it has also proven convenient at times, to refer to these arrangements of operations as modules, without loss of generality. The described operations and their associated modules may be embodied in software, firmware, hardware, or any combinations thereof.

Any of the steps, operations, or processes described herein may be performed or implemented with one or more hardware or software modules, alone or in combination with other devices. In some examples, a software module is implemented with a computer program object comprising a computer-readable medium containing computer program code, which can be executed by a computer processor for performing any or all of the steps, operations, or processes described.

Examples may also relate to an apparatus for performing the operations herein. This apparatus may be specially constructed for the required purposes, and/or it may comprise a general-purpose computing device selectively activated or reconfigured by a computer program stored in the computer. Such a computer program may be stored in a non-transitory, tangible computer readable storage medium, or any type of media suitable for storing electronic instructions, which may be coupled to a computer system bus. Furthermore, any computing systems referred to in the specification may include a single processor or may be architectures employing multiple processor designs for increased computing capability.

Examples may also relate to an object that is produced by a computing process described herein. Such an object may comprise information resulting from a computing process, where the information is stored on a non-transitory, tangible computer readable storage medium and may include any implementation of a computer program object or other data combination described herein.

The language used in the specification has been principally selected for readability and instructional purposes, and it may not have been selected to delineate or circumscribe the subject matter. It is therefore intended that the scope of this disclosure be limited not by this detailed description, but rather by any claims that issue on an application based hereon. Accordingly, the disclosure of the examples is intended to be illustrative, but not limiting, of the scope of the subject matter, which is set forth in the following claims.

Specific details were given in the preceding description to provide a thorough understanding of various implementations of systems and components for a contextual connection system. It will be understood by one of ordinary skill in the art, however, that the implementations described above may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

The foregoing detailed description of the technology has been presented for purposes of illustration and description. It is not intended to be exhaustive or to limit the technology to the precise form disclosed. Many modifications and variations are possible in light of the above teaching. The described embodiments were chosen in order to best explain the principles of the technology, its practical application, and to enable others skilled in the art to utilize the technology in various embodiments and with various modifications as are suited to the particular use contemplated. It is intended that the scope of the technology be defined by the claim.

What is claimed is:

1. A computer-implemented method, comprising:
receiving ambient and direct data corresponding to different sensors and devices within a multi-tenant environment, wherein the ambient and direct data is received through a gateway device implemented in the multi-tenant environment, and wherein the multi-tenant environment is associated with a set of users;
identifying one or more events occurring in the multi-tenant environment, wherein the one or more events are identified based on the ambient and direct data;
extracting attribute data from the ambient and direct data, wherein the attribute data corresponds to an individual detected within the multi-tenant environment, and wherein the individual is associated with the one or more events;
dynamically processing the attribute data through a trained classification algorithm to calculate an attribute vector, wherein attribute vectors are calculated to disambiguate the set of users according to a set of known attribute vectors associated with the set of users;
calculating match probability scores corresponding to the set of users by determining differences between values of the attribute vector and known values corresponding to the set of known attribute vectors;
identifying a user from the set of users based on the match probability scores, wherein a match probability score associated with the user satisfies a score threshold value;
associating the one or more events with the user; and
dynamically updating a known attribute vector associated with the user based on the one or more events and feedback corresponding to the one or more events, wherein the updated known attribute vector is used to better associate similar attribute data with the user.

2. The computer-implemented method of claim 1, further comprising:
updating the trained classification algorithm according to the feedback corresponding to the one or more events.

3. The computer-implemented method of claim 1, wherein the attribute data includes physical attribute data, activity data, location data, and timing data corresponding to the one or more events.

4. The computer-implemented method of claim 1, further comprising:
obtaining additional data associated with the user, wherein the additional data is obtained through one or more wearable devices and other personal devices associated with the user; and
aggregating the additional data and the attribute data.

5. The computer-implemented method of claim 1, further comprising:
dynamically converting new attribute data into a new attribute vector, wherein the new attribute data is extracted from new ambient and direct data obtained within the multi-tenant environment;
determining that new match probability scores associated with the new attribute vector fail to satisfy the score threshold value; and
associating a new event corresponding to the new attribute data with an unknown user.

6. The computer-implemented method of claim 1, further comprising:
generating one or more notifications based on the one or more events and identification of the user; and
transmitting the one or more notifications according to a set of configurations, wherein the set of configurations is defined based on the identification of the user.

7. The computer-implemented method of claim 1, wherein the different sensors and devices include a set of Internet-of-Things (IoT) devices implemented in the multi-tenant environment, and wherein the set of IoT devices is connected to the gateway device through one or more wireless communications channels.

8. A system, comprising:
one or more processors; and
memory storing thereon instructions that, as a result of being executed by the one or more processors, cause the system to:
receive ambient and direct data corresponding to different sensors and devices within a multi-tenant environment, wherein the ambient and direct data is received through a gateway device implemented in the multi-tenant environment, and wherein the multi-tenant environment is associated with a set of users;
identify one or more events occurring in the multi-tenant environment, wherein the one or more events are identified based on the ambient and direct data;
extract attribute data from the ambient and direct data, wherein the attribute data corresponds to an individual detected within the multi-tenant environment, and wherein the individual is associated with the one or more events;
dynamically process the attribute data through a trained classification algorithm to calculate an attribute vector, wherein attribute vectors are calculated to disambiguate the set of users according to a set of known attribute vectors associated with the set of users;
calculate match probability scores corresponding to the set of users by determining differences between values of the attribute vector and known values corresponding to the set of known attribute vectors;
identify a user from the set of users based on the match probability scores, wherein a match probability score associated with the user satisfies a score threshold value;
associate the one or more events with the user; and
dynamically update a known attribute vector associated with the user based on the one or more events and feedback corresponding to the one or more events, wherein the updated known attribute vector is used to better associate similar attribute data with the user.

9. The system of claim 8, wherein the instructions further cause the system to:
update the trained classification algorithm according to the feedback corresponding to the one or more events.

10. The system of claim 8, wherein the attribute data includes physical attribute data, activity data, location data, and timing data corresponding to the one or more events.

11. The system of claim 8, wherein the instructions further cause the system to:
obtain additional data associated with the user, wherein the additional data is obtained through one or more wearable devices and other personal devices associated with the user; and
aggregate the additional data and the attribute data.

12. The system of claim 8, wherein the instructions further cause the system to:
dynamically convert new attribute data into a new attribute vector, wherein the new attribute data is extracted from new ambient and direct data obtained within the multi-tenant environment;
determine that new match probability scores associated with the new attribute vector fail to satisfy the score threshold value; and
associate a new event corresponding to the new attribute data with an unknown user.

13. The system of claim 8, wherein the instructions further cause the system to:
generate one or more notifications based on the one or more events and identification of the user; and
transmit the one or more notifications according to a set of configurations, wherein the set of configurations is defined based on the identification of the user.

14. The system of claim 8, wherein the different sensors and devices include a set of Internet-of-Things (IoT) devices implemented in the multi-tenant environment, and wherein the set of IoT devices is connected to the gateway device through one or more wireless communications channels.

15. A non-transitory computer-readable storage medium storing thereon executable instructions that, as a result of being executed by one or more processors of a computer system, cause the computer system to:
receive ambient and direct data corresponding to different sensors and devices within a multi-tenant environment, wherein the ambient and direct data is received through a gateway device implemented in the multi-tenant environment, and wherein the multi-tenant environment is associated with a set of users;
identify one or more events occurring in the multi-tenant environment, wherein the one or more events are identified based on the ambient and direct data;
extract attribute data from the ambient and direct data, wherein the attribute data corresponds to an individual detected within the multi-tenant environment, and wherein the individual is associated with the one or more events;
dynamically process the attribute data through a trained classification algorithm to calculate an attribute vector, wherein attribute vectors are calculated to disambiguate the set of users according to a set of known attribute vectors associated with the set of users;
calculate match probability scores corresponding to the set of users by determining differences between values of the attribute vector and known values corresponding to the set of known attribute vectors;
identify a user from the set of users based on the match probability scores, wherein a match probability score associated with the user satisfies a score threshold value;
associate the one or more events with the user; and
dynamically update a known attribute vector associated with the user based on the one or more events and feedback corresponding to the one or more events, wherein the updated known attribute vector is used to better associate similar attribute data with the user.

16. The non-transitory computer-readable storage medium of claim 15, wherein the executable instructions further cause the computer system to:
update the trained classification algorithm according to the feedback corresponding to the one or more events.

17. The non-transitory computer-readable storage medium of claim 15, wherein the attribute data includes physical attribute data, activity data, location data, and timing data corresponding to the one or more events.

18. The non-transitory computer-readable storage medium of claim 15, wherein the executable instructions further cause the computer system to:
obtain additional data associated with the user, wherein the additional data is obtained through one or more wearable devices and other personal devices associated with the user; and
aggregate the additional data and the attribute data.

19. The non-transitory computer-readable storage medium of claim 15, wherein the executable instructions further cause the computer system to:
dynamically convert new attribute data into a new attribute vector, wherein the new attribute data is extracted from new ambient and direct data obtained within the multi-tenant environment;
determine that new match probability scores associated with the new attribute vector fail to satisfy the score threshold value; and
associate a new event corresponding to the new attribute data with an unknown user.

20. The non-transitory computer-readable storage medium of claim 15, wherein the executable instructions further cause the computer system to:
generate one or more notifications based on the one or more events and identification of the user; and
transmit the one or more notifications according to a set of configurations, wherein the set of configurations is defined based on the identification of the user.

21. The non-transitory computer-readable storage medium of claim 15, wherein the different sensors and devices include a set of Internet-of-Things (IoT) devices implemented in the multi-tenant environment, and wherein the set of IoT devices is connected to the gateway device through one or more wireless communications channels.
